(19) United States
(12) Patent Application Publication
(10) Pub. No.: US 2006/0206173 A1
(43) Pub. Date: Sep. 14, 2006

(54) Devices, Methods and Kits for Radiation Treatment via a Target Body Surface

(76) Inventors: Michael Gertner, Menlo Park, CA (US); Erica Rogers, Redwood City, CA (US)

Correspondence Address: WILSON SONSINI GOODRICH & ROSATI, 650 PAGE MILL ROAD, PALO ALTO, CA, 94304-1050, US

(21) Appl. No.: 11/276,787

(22) Filed: Mar. 14, 2006

Related U.S. Application Data
(63) Continuation-in-part of application No. 11/244,812, filed on Oct. 5, 2005.
(63) Continuation-in-part of application No. 11/152,946, filed on Jun. 14, 2005.
(60) Provisional application No. 60/690,792, filed on Jun. 15, 2005.
(60) Provisional application No. 60/661,688, filed on Mar. 14, 2005.

(51) Int. Cl. A61N 5/06

(52) U.S. Cl. 607/88

(57) ABSTRACT

A method and apparatus is described for treating a target body surface using a radiation applicator. The radiation applicator includes a radiation source in combination with a delivery applicator. The applicator has a low profile enable the patient to apply the applicator to a target area underneath, for example, clothing. The applicator can be configured to use one or more radiation sources to apply one or more types of radiation for one or more periods of time. Additionally, the applicator can be configured with a feedback loop to determine when a therapeutically desirable amount of radiation has been delivered.

### CROSS-REFERENCE

This application is also a continuation-in-part application of Ser. No. 11/244,812, filed Oct. 5, 2005, which claims benefit of U.S. Provisional Application No. 60/690,792, filed Jun. 15, 2005, which is incorporated herein by reference in its entirety and to which application priority is claimed under 35 USC § 120.

This application is also a continuation-in-part application of Ser. No. 11/152,946, filed Jun. 14, 2005, which claims benefit of U.S. Provisional Application No. 60/661,688 filed Mar. 14, 2005, which is also incorporated herein by reference in its entirety and to which application priority is claimed under 35 USC § 120.

### BACKGROUND OF THE INVENTION

1. Field of the Invention

The invention relates to devices and methods for delivering radiation to target body surfaces, including dermatologic treatment methods and apparatuses and ocular treatment methods and devices. The invention also relates to methods of manufacturing devices for delivering radiation to target body surfaces. Additionally, the invention includes methods and apparatuses for treating skin, including psoriasis, atopic dermatitis, contact dermatitis, vitiligo, seborrheic dermatosis and skin cancer; nails, and ocular disorders.

2. Background of the Invention

The therapeutic use of light has been shown to be effective in the treatment of various medical conditions. For example, whole body exposure to ultraviolet (“UV”) light has been used for medical applications, such as the treatment of psoriasis and vitiligo. Ultraviolet lasers and lamps have also been designed to illuminate more localized regions of the skin for treatment of lesions and marks.

It is estimated that 5 million adults in the U.S. suffer from psoriasis. Psoriasis is broken into five categories: plaque, guttate, inverse, pustular, and erythrodermic. Psoriasis can occur at a variety of locations, including hands, feet, scalp, etc. For example, plaque psoriasis, the most common form, is characterized by raised, inflamed, red lesions covered by a silvery white scale. It is typically found on the elbows, knees, scalp and lower back, although it can occur on any area of the skin. The guttate form of psoriasis resembles small, red, individual spots on the skin. Guttate lesions usually appear on the trunk and limbs. These spots are not normally as thick or as crusty as lesions of plaque psoriasis.

Vitiligo is a pigmentation disorder in which melanocytes (the pigment making cells in the skin), the mucous membrames, and the retina are destroyed. As a result, white patches of skin appear on different parts of the body. It is estimated that 1-2% of the world's population (40-50 million people) have vitiligo, 2-5 million of whom are located in the United States.

Atopic dermatitis, more commonly referred to as eczema, is a chronic (long-lasting) disease that affects the skin. In atopic dermatitis, the skin becomes extremely itchy. Scratching leads to redness, swelling, cracking, “weeping” clear fluid, and finally, crusting and scaling. It is estimated that 5 million Americans under the age of 65 have atopic dermatitis. Seborrheic dermatitis is a chronic form of dermatitis characterized by oily scales, crusty yellow patches, and itching that occurs primarily on the scalp and face. Contact dermatitis is a disorder which can be allergic or non-allergic in nature but is almost always immune mediated and affects several million people in the United States alone. It is considered a major cause of occupational disability and leads to billions of dollars in health care expenditures.

It is estimated that more than 1 million people are diagnosed with skin cancer each year; and 1 in 5 Americans will have an incident of skin cancer in their lifetime. Skin cancers are categorized as: melanoma, basal cell carcinoma, squamous cell carcinoma, and actinic keratosis, Melanoma is the most serious form of skin cancer, but if treated early can be cured. Basal cell carcinoma is the most common type of skin cancer. Skin cancer can be treated by, among other things, radiation therapy.

A variety of devices are known for delivering light and/or radiation. For example, PCT Publication WO 03/013653 to Kemeny et al. for Phototherapeutical Apparatus (see also, U.S. Patent Pub. US 2004/0030368 to Kemeny et al. for Phototherapeutical Method and System for the Treatment of Inflammatory and Hyperproliferaitve Disorders of the Nasal Mucosa; WO 2005/000389 to Fiset for Skin Tanning and Light Therapy Incorporating Light Emitting Diodes (see also, U.S. Patent Pub. 2004/0232339 to Lanoue for Hyperspectral Imaging Workstation Having Visible/Near-Infrared and Ultraviolet Image Sensors). U.S. Pat. No. 6,290,713 to Russell for Flexible Illuminators for Phototherapy; U.S. Patent Pub. 2004/0176824 to Weckworth for Method and Apparatus for the Repigmentation of Human Skin; U.S. Pat. No. 6,730,113 to Eckhardt et al. for Method and Apparatus for Sterilizing or Disinfecting A Region Through a Bandage; U.S. Pat. No. 6,096,066 to Chen et al. for Conformal Patch for Administering Light Therapy to Subcutaneous Tumors; and U.S. Pat. No. 6,645,230 to Whitehurst for Therapeutic Light Source and Method. A variety of devices are also known for providing bandages or dressing, including, for example, U.S. Pat. No. 2,992,644 to Plantinga et al. for Dressing; U.S. Pat. No. 3,416,525 to Yeremian for Stabilized Non-Adherent Dressing; U.S. Pat. No. 3,927,669 to Glatt for Bandage Construction; U.S. Pat. No. 4,126,130 to Cowden for Wound Protective Device; U.S. Pat. No. 4,561,435 to McKnight et al. for Wound Dressing; U.S. Pat. No. 4,616,644 to Saferstein et al. for Hemostatic Adhesive Bandage; U.S. Pat. No. 4,671,266 to Lengyel et al. for Blister Bandage; U.S. Pat. No. 4,901,714 to Jensen for Bandage; U.S. Pat. No. 5,336,209 to Porilli for Multi-Function Wound Protection Bandage and Medicant Delivery System with Simultaneous Variable Oxygenation; U.S. Pat. No. 5,954,679 to Baranitsky for Adhesive Bandage; U.S. Pat. No. 6,343,604 B1 to Beall for Protective Non Occlusive Wound Shield; U.S. Pat. No. 6,384,294 B1 to Levin for Protective Bandages Including Force-Transmission-Impeding Members Thereof; and U.S. Patent Publications US 2001/0028943 A1 to Mashiko et al. for Adhesive Film for Adhesive Bandage Using Said Adhesive Film; US 2002/0128580 A1 to Carlson for Self-Adhering Friction Reducing Liner and Method of Use; US 2002/0183813 A1 to Augustine et al. for Treatment Apparatus with a Heater Adhesively Joined to the Bandage; US 2003/0199800 A1 to Levin for Bandage Including Perforated Gel; US 2003/0163074 A1 to McGowan et al. for Wound Dressing Impervious to Chemical and Biological Agents; US 2003/0143264 A1 to Margiotta for Topical Anesthetic-Antiseptic Patch; US 2004/0087884 A1 to Haddock et al. for Textured Breathable Films and Their Use as Backing Material for Bandages; US 2004/0049144 A1 to Cea for Hypoallergenic Bandage; US2004/0260365 to Groseth et al. for Photodynamic Therapy Lamp; and US 2005/0010154 A1 to Wright et al. for Adhesive Bandage for Protection of Skin Surface.

### SUMMARY OF THE INVENTION

The invention relates to a photodynamic or radiation treatment apparatus having a plurality of light and/or radiation source(s) adapted to irradiate a target portion of a body.

An embodiment of the invention includes a photodynamic treatment apparatus comprising: a plurality of light sources adapted to emit light at a peak wavelength between about 300 nm and about 320 nm; a light source support structure adapted to be worn by a patient and to support the light sources in a manner permitting light from the light sources to reach a skin area of the patient; a controller adapted to operate the light sources; and a power supply adapted to provide power to the light sources. The controller can be configured to control the operation of the light sources automatically or semi-automatically.

An embodiment of the invention includes a phototherapeutic treatment apparatus comprising: one or more light sources adapted and configured to emit light at one or more peak wavelengths in a target range; a light source delivery structure adapted and configured to be worn by a patient and to support the light sources in a manner permitting light from the light sources to reach a target body surface of the patient; a controller adapted to operate the light sources; and a power supply adapted to provide power to the light sources.

Another embodiment of the invention includes a radiation treatment device adapted to delivery radiation therapy to a target body surface comprising: a substrate adapted to contact a target surface of a human body; a first radiation source for delivering radiation to the target surface area of the human body and adapted to engage the substrate; and a controller integrated with the substrate and adapted to control at least one of duration or amount of radiation delivered by the radiation source.

The radiation applicator may include one or more radiation source(s) coupled to a substrate (e.g., a layer, such as a fabric strip on which radiation source(s) are positioned for presentation of radiation to a target body surface). In one embodiment, a controller and a power source are also coupled to a substrate. In another embodiment, the size of the controller and the power source are such that the radiation applicator is portable and comfortable to wear without, for example, being held in the hand of a patient. In an embodiment, the radiation applicator is self-contained and does not need to be attached to any external, non-portable, device to operate.

In yet another embodiment, the substrate (supporting the radiation source(s)) is flexible. In an embodiment, the radiation applicator is worn like a bandage and the radiation source(s) are located in and/or on a region of the substrate that is smaller than the entire substrate (analogous in structure to the gauze pad of a Bandaid® type bandage, for example). Further, the substrate can include an adhesive section to conformably retain the radiation applicator to the target body surface. In yet another embodiment, the size of the region containing the radiation source(s) is based on an expected size of a typical disorder being treated, while, in contrast, the size of the entire substrate is based on the minimum size that is large enough to ensure that the radiation applicator is securely attached to the body being treated.

In still other embodiments, any of the radiation source(s) can be turned on and off such that at any one time the number of radiation source(s) that is on is less than the entire number of radiation source(s) presented on the substrate. In an embodiment, the duty cycle used and the number of radiation source(s) that are on at any one time are based on the power capabilities of the power source. In an embodiment, the duty cycle and number of radiation source(s) that are on are based on cooling requirements of the radiation source(s), a prescribed treatment, and/or a temperature range that the patient is expected to find comfortable. In another embodiment, some of the radiation source(s) are not turned on at all as programmed by the patient or medical practitioner to provide a desired sequence of operations because, for example, the area of required treatment of the body is smaller than the area of the radiation source(s). The radiation applicator may also have a calibration mode for calibrating the radiation dose to a specific body and/or patient, or to provide a change in radiation intensity along the substrate.

In yet another embodiment of the invention a method of providing light therapy to a skin area of a patient comprising: attaching a plurality of light sources to the skin area; providing power to the light sources from a power supply worn by the patient; and providing light having a peak wavelength between about 300 nm and about 320 nm from one or more of the light sources to the skin area is provided. Embodiments of the method can include providing light therapy to a skin area of a patient wherein the step of providing light comprises providing light having a peak wavelength between about 308 nm and about 312 nm from one or more of the light sources to the skin area. In other embodiments of the method light therapy is provided to a skin area of a patient from first and second sets of one or more light sources, step of providing light comprises providing light from the first set of light sources and not from the second set of light sources and providing light from the second set of light sources and not from the first set. Additional methods can include providing light from the first set of light sources comprises supplying an energy dose of between about 100 mJ/cm² and about 600 mJ/cm² from the first set of light sources. While performing these methods, the duration of providing light can also be monitored. Additionally, the light source can be calibrated to provide a therapeutic dose of energy.

In yet another embodiment of the invention a method of calibrating a target surface area of a human body comprising: applying a radiation treatment device to the target area of the human body; monitoring an irradiation of the target surface area of the human body; assessing whether erythema is present; and setting a minimum erythemal dosage for the radiation treatment device is provided.

In yet another embodiment of the invention, a method of calibrating a target surface area of a human body comprising: applying a radiation treatment device to the target area of the human body; monitoring a condition of the target surface area of the human body; assessing whether erythema is present; and setting a minimum erythemal dosage for the radiation treatment device is provided.

In still another embodiment of the invention, a method of treating a target surface area of a human body comprising: applying a radiation treatment device to the target area of the human body; and monitoring the irradiation of the target surface area of the human body is provided.

A kit for treating a target area of a human body needing therapy is also provided. The kit comprising: a first radiation treatment device having a substrate adapted to contact a target surface of a human body, a radiation source for delivering radiation to the target surface area of the human body and adapted to engage the substrate, and a controller adapted to control at least one of duration or amount of radiation delivered by the radiation source; and a second radiation treatment device having a substrate adapted to contact a target surface of a human body, a radiation source for delivering radiation to the target surface area of the human body and adapted to engage the substrate, and a controller adapted to control at least one of duration or amount of radiation delivered by the radiation source.

An embodiment of the invention includes a phototherapeutic treatment apparatus comprising: a first light source adapted to emit light at a first peak wavelength between about 250 nm and about 320 nm; a second light source adapted to emit light at a second peak wavelength the same or different than the first peak wavelength; a light source support structure adapted to be applied to a patient and to support the light sources in a manner permitting light from the light sources to reach a prescribed body surface of the patient; a controller adapted to automatically or semi-automatically operate the light source within safe and therapeutic limits; and a power supply adapted to provide power to the light sources through the controller. In some embodiments, the invention includes a light source adapted to deliver a minimal erythema dose. In other embodiments, the apparatus is adapted to provide a programmable light source adapted and configured to deliver light to a prescribed area, to turn off after delivery of a prescribed dose, and/or to monitor the radiation device. In still other embodiments, the light source is adapted and configured to deliver light, the light being selected from group consisting of UVA, blue, yellow, white, and infrared. In yet other embodiments, the light source is adapted and configured to deliver intense pulsed white light. Additionally, the light source can be adapted and configured to deliver intense pulsed white light in combination with infrared light or any other desired combination of light

Another embodiment of the invention includes a phototherapeutic treatment apparatus comprising: a light source adapted to emit light at a peak wavelength between about 250 nm and about 320 nm; a light source support structure adapted to be externally applied to a patient and to support the light sources in a manner permitting light from the light sources to reach a target body surface of the patient; a controller adapted to operate the light source; and a wearable power supply adapted to provide power to the light sources. In some embodiments, the invention includes a light source adapted to deliver a minimal erythema dose. In other embodiments, the device is adapted to provide a programmable light source adapted and configured to deliver light to a prescribed area, to turn off after delivery of a prescribed dose, and/or to monitor the radiation device. In still other embodiments, the light source is adapted and configured to deliver light, the light being selected from group consisting of UVA, blue, yellow, white, and infrared. In yet other embodiments, the light source is adapted and configured to deliver intense pulsed white light or any other desired combination of light. Additionally, the light source can be adapted and configured to deliver intense pulsed white light in combination with infrared light.

Yet another embodiment of the invention includes a phototherapeutic treatment apparatus comprising: a light sources adapted to emit light at a peak wavelength between about 255 nm and about 320 nm; a light source support structure adapted to be externally applied to a patient and to support the light sources in a manner permitting light from the light sources to reach a target body surface of the patient; a controller adapted to operate the light sources; and a power supply adapted to provide power to the light sources.

A method according to one embodiment of the invention includes treating a target body surface comprising the steps of: applying a radiation therapy device adapted and configured to include a wearable power supply, a radiation source, and a controller to the target body surface; delivering radiation from the radiation source to a first portion of the target body surface; and controlling a radiation output from the radiation source to a first portion of the target body surface in relation to a therapeutic dose during the step of delivering radiation. The method can be performed using a suitable radiation source such as an LED, or a UV LED. In some embodiments the method is performed by relating the therapeutic dose to minimal erythema dose. Additionally, the step of controlling the radiation dose can include turning-off the radiation dose; and/or can include turning on the radiation dose. The radiation therapy device can be a wearable article or any device adapted and configured to apply radiation or light to a target body surface. Further, the step of delivering radiation can include delivering a timed radiation dose. The radiation dose can be delivered to any portion of the target body surface between 100 mJ/cm and 3 J/cm². In some instances, it may be desirable to deliver radiation doses to a portion of the target body surface at a wavelength between 295 and 315 nm. In other embodiments, the radiation dose delivered to any portion of the target body surface has a first wavelength between 295 and 315 nm and a second wavelength between 340 and 400 nm. In still other embodiments, the radiation dose delivered to any portion of the target body surface has as wavelength greater than about 700 nm. The method can also include the step of administering a photosensitizing agent. Additionally, the method can include the step of programming the radiation device to apply radiation to a prescribed area. In some embodiments, the method can further comprise the step of programming the radiation device to turn off after a prescribed dose is applied. Further, the step of monitoring the radiation device can be included in the method, as desired. The radiation dose delivered to any portion of the target body surface can be selected from: infrared light, intense pulsed light, white light, and combinations thereof. Radiation sources suitable for any of the methods include, for example, radiation sources configured to deliver UVA, blue, yellow, white and infrared light. The method can also include delivering more than one light, pulsed light, etc., as desired. Further, the device an be programmed to deliver light from a subset of the device, such as form an area of the device corresponding to prescribed area to be treated.

Another embodiment of the invention includes a method of treating a target body surface comprising the steps of: applying a radiation therapy device adapted and configured to include a wearable power supply, a radiation source, and a controller to the target body surface; delivering radiation from the radiation source to a first portion of the target body surface; controlling a radiation output from the radiation source to a first portion of the target body surface in relation to a therapeutic dose during the step of delivering radiation; decreasing the radiation dose to the first portion of the target body surface; and increasing the radiation dose to a second portion of the target body surface. According to an embodiment of the method, the radiation source can be an LED. Suitable LEDs can be configured to deliver, for example, UVA, blue, yellow, white and infrared light. UV LEDs that deliver a peak wavelength between 250 nm and 320 nm are also suitable. When performing the method, the minimal erythema dose can be related or correlated to a therapeutic dose. Further the device can be adapted to decrease or turn-off the radiation source or increase or turn on the radiation source to change the radiation dose provided. The device can be a wearable article or any device adapted and configured to apply radiation or light to a target body surface. Under some embodiments of the methods, the radiation dose delivered to any portion of the target body surface is between 1 mJ/cm² and 3 J/cm². In other embodiments, the radiation output delivered to any portion of the target body surface has a wavelength between 295 and 315 nm, 340 and 400 nm or greater than about 700 nm. In some embodiments, it may be desirable to administer a photosensitizing agent. In other embodiments, the radiation device is programmed to apply radiation to a prescribed area, or to turn off after a prescribed dose is applied. In other embodiments, the radiation device is monitored. In still other embodiments, the radiation dose delivered to any portion of the target body surface is infrared light, pulsed white light, white light, or any other suitable light. Additionally, in some embodiments, the light delivered can be a combination of lights, such as white light in combination with infrared light.

A phototherapeutic treatment device comprising: a plurality of light sources adapted to emit light at a peak wavelength; a support structure adapted to be applied to a patient and further adapted to support the light sources in a manner permitting light from the light sources to reach a prescribed body surface of the patient; and a controller adapted to variably activate a subset of the plurality of light sources.

In still another embodiment of the invention, a kit is provided for delivering therapy to an eye. The kit comprising: a contact lens with a light blocking portion; a phototherapeutic treatment apparatus comprising a light source adapted to emit light at a peak wavelength; a light source support structure adapted to support the light sources in a manner permitting light from the light sources to reach an area of the patient; and a power supply adapted to provide power to the light sources. The light source provided in the kit can be adapted and configured to emit ultraviolet light and block a portion blocks ultraviolet light.

### INCORPORATION BY REFERENCE

All publications, patents and patent applications mentioned in this specification are herein incorporated by reference to the same extent as if each individual publication, patent or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates an example of a radiation applicator for applying radiation to a target surface;

FIG. 2A illustrates an example of target surface of a body being treated using the radiation applicator of FIG. 1; FIG. 2B illustrates a cross-sectional view of a target surface of a body being treated using the radiation applicator of FIG. 1;

FIG. 3 illustrates a block diagram of an example of the radiation applicator of FIG. 1;

FIG. 4 illustrates a block diagram of a controller;

FIG. 5A shows a block diagram of an example of a radiation source used in FIGS. 1-3; FIG. 5B illustrates a cross section of a radiation applicator of FIG. 3; FIG. 5C illustrates another example of a radiation source of FIG. 1; FIG. 5D is a close-up of a molded covering with optical components built in; and FIG. 5E is a close-up of a mount with three-dimensional geometries optimized for radiation extraction from the source.

FIG. 6A illustrates yet another example of a radiation applicator; FIG. 6B illustrates an example of a cross-section of the radiation applicator of FIG. 6A; FIG. 6C illustrates a radiation applicator delivering radiation therapy to a prescribed surface area within a target body surface;

FIGS. 7A-B illustrates another embodiment of an optical therapy devices for treating a target surface where the target surface is the eye;

FIG. 8A illustrates an optical therapy device adapted and configured to deliver optical therapy to a patient's skin; FIG. 8B illustrates a wearable optical therapy device in the form of a wrist bracelet; FIG. 8C illustrates an optical therapy device adapted and configured to deliver optical therapy to a patient's fingernail; FIG. 8D illustrates an optical therapy device in the form of an adhesive bandage;

FIG. 9 illustrates a flowchart of an a method of calibrating the radiation applicator for a particular patient;

FIG. 10 illustrates a flowchart of an example of a method of using the radiation applicator;

FIG. 11 illustrates a flowchart of an example of a method of assembling the radiation applicator;

FIG. 12 depicts a detailed view of a module which is insertable into a radiation applicator; and

FIG. 13 illustrates a flowchart of a method for providing light therapy.

### DETAILED DESCRIPTION OF THE INVENTION

A radiation applicator used for irradiating a target portion of a body for medical treatment is disclosed. In an embodiment, radiation delivered by a radiation applicator is ultraviolet light. In other embodiments, other forms of radiation may be delivered by the radiation applicator.

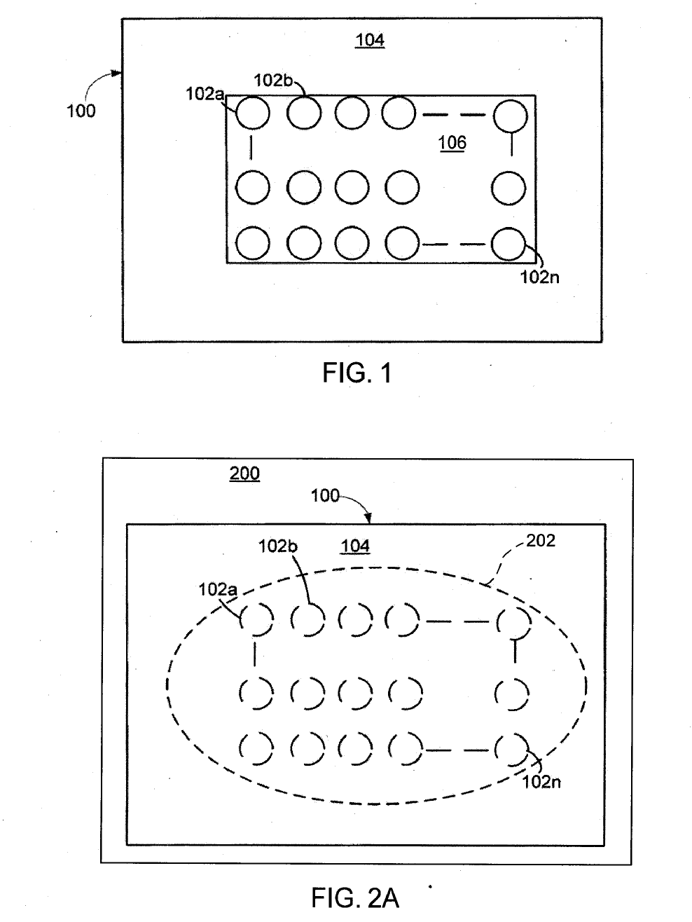

FIG. 1 shows a radiation applicator 100 for treating a target surface of a body with radiation. As will be appreciated by those skilled in the art, the target surface of a body includes the portion of a body surface onto which a radiation applicator is applied. At least a portion of the target body surface will include an area to which radiation therapy will be applied, such as a lesion. The portion of the target body surface to which radiation therapy is applied can be referred to as the therapeutic surface area or the prescribed surface area. As will further be appreciated by those of skill in the art, the therapeutic surface area can be of a size and shape that may or may not be conform with the size and shape of the area comprising the target body surface. Thus, the size and shape of both the therapeutic or prescribed surface area can be the same, or substantially the same, as the size and shape of the target body surface. Alternatively, the size and shape of the therapeutic or prescribed surface area can be smaller or larger than the target body surface, without departing from the scope of the invention.

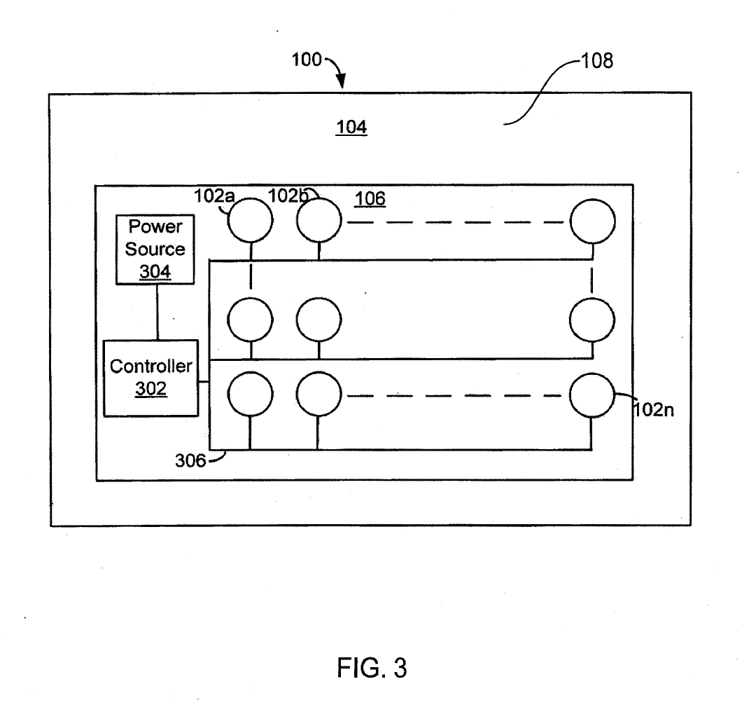

The radiation applicator 100 has at least a first side and a second side, or a top side and a bottom side with one side applied to the target body surface while the other side, typically, is not. The target surface is typically an exposed portion or surface, e.g. of skin, where it is desirable to apply radiation. Radiation applicator 100 may include one or more radiation source(s) 102 (e.g. 102a-102n) each of which has at least a first side and a second side, and substrate 104, also having a first side and a second side, which can be in the form of a layer or material on which the electrodes are formed or fabricated. In a preferred embodiment a plurality of radiation sources 102 are provided. Radiation sources refers to the actual source of the radiation and can also include structural elements associated with the source of energy which allow the radiation source to be manipulated independently of the substrates and other radiation sources. For example, (as discussed below) in the case where the radiation source is a light source, radiation source 102 can include a header, electrodes, reflecting features, focusing features, mounts with circuits and/or heat transferring features included thereon, and submounts. In further embodiments, the radiation applicator 100 has a region 106 that has a surface area smaller than the surface area of the substrate 104 (as illustrated in FIG. 3). As will be appreciated by those skilled in the art, radiation applicator 100 need not have all of the components depicted in FIG. 1 and/or may include other components in addition to or instead of those depicted with FIG. 1. For purposes of illustration, the geometric profile of the radiation applicator 100 has been shown as having a rectangular profile (e.g. a length greater than a width). As will be appreciated by those skilled in the art, other profiles can be employed, either geometric or non-geometric (e.g., random) without departing from the scope of the invention. The various layers and elements of the applicator 100 can be configured such that each provides a surface-to-surface contact with an adjacent layer and/or element.

Radiation source(s) 102 may produce any of a variety of types of radiation, such as UV light, white light, and/or infrared light that are used for treating disorders, ailments or diseases by irradiating a target portion of the body, such as an exposed surface of skin. A variety of dermatologic conditions, such as psoriasis, contact dermatitis, atopic dermatitis, vitiligo, seborrheic dermatosis, acne, cellulite, unwanted hair, unwanted blood vessels, and skin cancer, may be treated with various wavelengths of light, as discussed above. For example, when treating psoriasis, radiation source(s) 102 may emit light having a wavelength in the UVB range, including 295-320 nm, 300-305 nm, 308-315 nm, or a combination of these wavelengths in one or more peaks. When treating psoriasis with psoralens (PUVA), it is desirable to use radiation sources which emit light in the UVA range. For example, between 320 nm and 340 nm, between 341 nm and 360 nm, and/or between 361 nm and 390 nm. Additionally, there may be any number of radiation source(s) 102 with any combination of wavelengths.

It may be desirable to provide radiation source(s) that are capable of delivering more than one type of radiation. For example, atopic dermatitis can be treated with a device using, for example, a combination of UVB and UVA wavelengths. Thus, alternatively, it may be desirable to provide radiation source(s) 102 within the substrate 104 that can deliver a first radiation type or wavelength in combination with radiation source(s) 102 that can deliver a second, or subsequent, radiation type or value that is different from the first radiation type or wavelength. As will be appreciated by those of skill in the art, additional wavelengths or sources of radiation can be included without departing from the scope of the invention, and thus the invention is not limited to the delivery of two radiation types.

Infectious disorders can also be treated with the radiation source(s). For example, where infectious disorders are treated, shorter wavelengths, including those having a wavelengths in the range 254-270 nm or 270-295 nm, have been shown to be beneficial. As will be appreciated, the various dashed lines between various ones of radiation source(s) 102 (e.g. 102a-102n) indicate that there may be any number of radiation source(s) in that location spanning the region of the dashed lines and the region between the dashed lines, as necessary or desirable.

In another embodiment, radiation source(s) 102 (e.g. 102a-102n) produce white light (500-750 nm), infrared light, microwaves, radiofrequency radiation, and/or other electromagnetic wavelengths, for example, or combinations thereof. Heat (via infrared light) sometimes promotes healing of sprains and muscle injuries, and additionally may produce a feeling of well-being, even if no actual healing occurs. Infrared wavelengths include wavelengths from 780 nm to 10 microns. Infrared light can also be used to aid in healing of open surface wounds on a body or to increase the blood flow to a body surface. In some embodiments, the infrared light can be used to increase local blood flow to a body surface in order to improve the efficacy of phototherapy or photodynamic therapy. In some embodiments, infrared light can be used to destroy hair follicles which results in permanent or semi-permanent hair removal; cellulite can also be treated with infrared wavelengths. Other wavelengths of light in the mid-visible range (e.g. about 500-650 nm) can be used to treat acne, wrinkles, or other undesirable spots; white light wavelengths can also be used for photorejuvenation and/or cellulite removal. Some wavelengths of light (e.g. those having a wavelength of 450-460 nm) may be effective in treating different disorders, such as for lowering the bilirubin count in babies. In one embodiment, radiation source(s) 102 are used for treating disorders on a surface of a body. In another embodiment, radiation source(s) 102 emit forms of radiation (e.g., wavelengths of light) that penetrate below the surface of the body, and radiation source(s) 102 are used for treating disorders below the surface of the body. In some embodiments, some of radiation source(s) emit forms of radiation that penetrate to difference levels than other of the radiation source(s) 102. In some embodiments, photodynamic therapy is initiated with radiation source(s) 102. Photosensitizers allow for the application of almost any wavelength. For example, a photosensitizer can be applied to a skin lesion, and then the radiation device can then be applied over the lesion for a long period of time, for example by bringing the device into nearness or contact with the skin, or by putting the device on the skin, where the time is sufficient for a requisite dose of radiation to treat the lesion. In the case where the device is portable, a patient does not have to wait in a physician's office and a physician does not have to spend valuable time manually applying a tedious treatment. Photodynamic therapy can include a portable light source (e.g. device 100) and a photosensitizer which can be administered systemically or injected into a lesion or placed in close proximity to the lesion (e.g. a cream). For example, the photosensitizer can be applied and then the radiation applicator applied to the area over time to activate the photosensitizer. Alternatively, the radiation device releases photosensitizer from a reservoir or from the substance of the device itself. For example, levulin is a photosensitizer used in combination with yellow light for photorejuvenation therapy.

In one embodiment, all radiation source(s) 102 produce the same peak wavelength and/or spectrum of radiation when activated. In another embodiment, different ones of radiation source(s) 102 produce different spectrums of radiation and/or have different peak wavelengths. In an embodiment, whether or not all radiation source(s) 102 are the same or some are different from others, the spectrum of radiation produced may be controllable (e.g., by adjusting the current) so that the wavelength or combination of wavelengths of light may be adjusted according to the type of disorder being treated. In some embodiments where an optical disperser is used, a multiplicity of radiation source(s) can be combined into a predetermined spectral output. In these embodiments, the spectrum can be tailored by turning one or more of the radiation sources on or off at different times.

Radiation source(s) 102 may require a power source. Embodiments including a power source are discussed in conjunction with FIGS. 3, 5C, and 6A, for example. Power sources may be portable (e.g. wearable or incorporated into the device, etc.) or non-portable (e.g. table top, wall-plug, or other wise connected to the device via cord, etc.) Alternatively, some radiation source(s) 102 may not require a power source. For example, radiation source(s) 102 may produce light via fluorescence or chemical luminescence. In another embodiment, radiation source(s) 102 can be powered by photovoltaic cells. Alternatively, radiation source(s) 102 may include a radioactive material that emits alpha, beta, and/or gamma particles. For example, radiation source(s) 102 may be discs of P-32, In-111, radioactive isotopes, Cesium 137 and/or another radioactive material, which may be useful for treating certain types of cancer. Additional radiation sources can include microwave emitters, electromagnetic emitters, and radiofrequency emitters.

Substrate 104 may take many forms. Substrate 104 may be any suitable material such as a piece of material, which in turn may be a strip of fabric. Substrate 104 may be solid, a mesh, or netting, for example. Substrate 104 may be a flexible material that can be wrapped around a limb or placed on another body part. In one embodiment, substrate 104 is a bandage. For example, substrate 104 may have an adhesive layer on at least a portion of one surface of the substrate such as the surface that contacts the target body surface. Alternatively, substrate 104 does not have an adhesive layer. In another embodiment, substrate 104 may be an article of clothing, such as a sock, a glove, a sweater, a ski mask, a headband, an arm band, a leg band, etc. In some embodiments, the substrate 104 is patient compatible. If substrate 104 is not patient compatible, then the substrate can be furthered covered with a patient compatible material. As will be appreciated by those skilled in the art, substrate 104 can be any material, surface or device adapted and configured to deliver radiation therapy to a body surface.

In another embodiment, instead of being flexible, substrate 104 is rigid and is held onto the portion of the body being treated by being attached to a bandage or by being wrapped within a bandage. Whether substrate 104 is rigid or flexible, a separate substrate, such as a stocking, a glove, or a circumferential cloth, may be utilized to hold the substrate 104 onto a target portion of a body.

Substrate 104 may be opaque, transparent, translucent, reflective, or made from a light scattering material. Radiation source(s) 102 (e.g. 102a-102n) may be located on substrate 104. For example, radiation source(s) 102 may be attached to a surface of substrate 104 and/or formed integrally within substrate 104 (e.g., embedded or formed within the substrate to provide a complete, unified radiation applicator 100). Alternatively, one portion of the radiation source can be attached on the outside of the material (e.g. the side of the material not facing the lesion or target body surface) and the other side of the radiation source (e.g. the light emitting side) is attached on the inside of the substrate (e.g. the side of the material facing the lesion). In this embodiment, the housing of the radiation source traverses the substrate 104 and the power is supplied along the surface of the substrate 104 facing away from the region of the body with the lesion. Substrate 104 may be of a size and/or shape that facilitates securely attaching radiation applicator 100 to a body. In an embodiment, radiation applicator 100 can be worn by a patient without any external attachments. In an embodiment, radiation applicator 100 may be self-contained. Making radiation applicator 100 self-contained and/or wearable without any external attachments (e.g., in the form of an adhesive bandage) facilitates making radiation applicator 100 portable. A portable applicator which can be worn by a patient under other clothes or while he or she is performing other tasks or while sleeping may have many advantages in terms of, for example, the quality of life of the patient and in terms of compliance.

Region 106 is a region of substrate 104 within which radiation source(s) 102 (e.g. 102a-102n) are located. Region 106 can have a surface area that is less than the surface area of substrate 104. Substrate region 106 may be of a size and/or shape that is expected to cover all of, or a substantial part of, a portion (of a body) affected by a typical occurrence of a particular type of disorder (such as a lesion). Alternatively, region 106 may be of a size and/or shape that is expected to be smaller than the portion of the body affected by a typical occurrence of a particular type of disorder. In one embodiment, substrate region 106 is defined only by the location of radiation source(s) 102, but is otherwise structurally identical to the rest of substrate 104. In another embodiment, region 106 may have one or more structural features that distinguish region 106 from the rest of substrate 104. In one example, substrate 104 is rectangular in shape, optionally having rounded corners, and region 106 is located in a central portion of substrate 104 that extends nearly the entire width of substrate 104, but only extends less than one third or less than one quarter of the length of the substrate 104. In a further embodiment of this example, substrate 104 is flexible and has an adhesive in the portions 108 outside of the region 106 for adhering to a body being treated, but no adhesive is inside of region 106. Region 106 may be analogous in structure to the gauze pad of a Bandaid® type bandage. In this example, region 106 and substrate 104 are of a similar size as the gauze pad region of a bandage for covering a cut or scrape. For example, region 106 may include a gauze pad, and any one of, any combination of, or all of radiation source(s) 102, controller 302 (discussed below), and/or power source 304 (discussed below) may be located on, behind, and/or embedded within the gauze pad.

As will be appreciated by those skilled in the art, the controller can be adapted and configured to control the delivery of radiation either automatically (i.e., without user intervention) or semi-automatically (with minimal or limited user intervention). The controller can be adapted and configured to control the amount of radiation delivered, the time for which radiation is delivered and the type of radiation delivered. Further, the controller can be adapted and configured to provide a therapeutic regimen, e.g. by altering or changing the type and/or amount of radiation delivered. The controller can also be adapted to dynamically control the therapeutic regimen delivered in response to feedback, as will be appreciated based on the teachings herein.

Substrate region 106 may include a protective layer for radiation source(s) 102 that is not present in the remainder of substrate 104. Within region 106, substrate 104 may have additional elements or features, such as structural features, that promote cooling, or condition the spectral output of radiation source(s) 102; for examples substrate 104 can contain a deposited reflective layer such as aluminum in the case of UV light. Alternatively, substrate 104 contains surface features which increase the surface area to promote heat transfer. Other elements and features include, but are not limited to, selectively providing perforations (not shown) that penetrate all or a portion of the radiation applicator 100 on at least a portion of the applicator. In yet another embodiment, region 106 may be a piece of removable material that supports radiation source(s) 102. Having a removable substrate region 106 allows the same substrate 104 to be used with a multiplicity of different sets of radiation source(s) 102 in which each set is designed for treating a different disorder or set of disorders. In another embodiment, a material covers region 106. This material is a disposable material which is transparent to the radiation from radiation source(s) 102 and is discarded after the therapy, allowing the devices in region 106 to be reusable without concern for the devices being soiled. In another embodiment, substrate region 106 may be absent, and radiation source(s) 102 may be uniformly distributed throughout substrate 104.

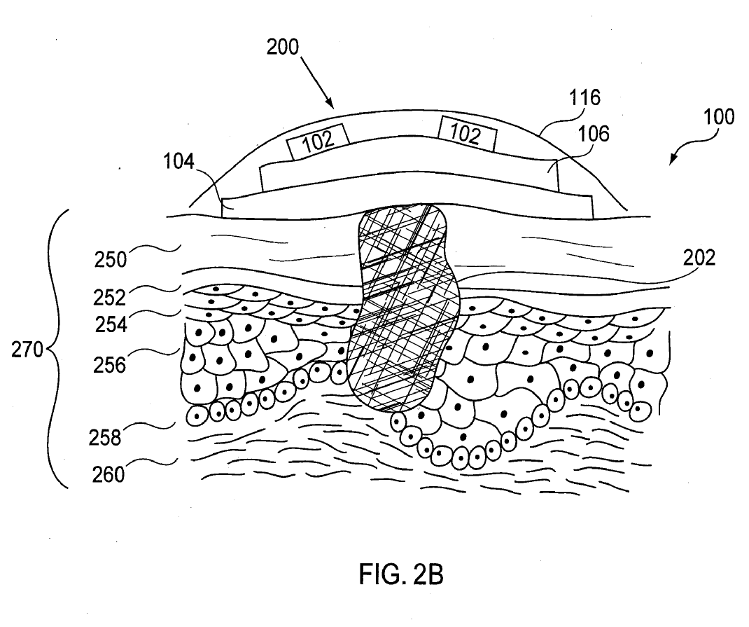

FIG. 2A shows an example of a portion of a body 200, e.g. a target portion of a human body, such as a skin layer, while being treated. During treatment of body portion 200, radiation applicator 100 is placed on a lesion 202 on body portion 200. Lesion 202 can be any patch of unhealthy or unwanted tissue surface that is expected to be at least partially treatable by irradiating with radiation, such as light. (Lesion 202 is illustrated with a dashed line in FIG. 2A because lesion 202 is under radiation applicator 100 and specifically under region 106.) Body portion 200 is any target surface of a body, e.g., external, internal, or externally/internally exposed, portion of the body such as skin. For example, portion 200 may be a portion of skin on a limb (e.g., the arm), or the hand of a patient. In the embodiment of FIG. 2A, substrate 104 is a single opaque layer and radiation source(s) 102 (e.g. 102a-102n) are placed on one side of substrate 104. Consequently, radiation source(s) 102 (e.g. 102a-102n) are drawn with dashed lines to indicate that radiation source(s) 102 are between substrate 104 and lesion 202, so as to irradiate lesion 202 without being impeded by substrate 104. Similar to FIG. 1, the various dashed lines between radiation source(s) 102 indicate that there may be any number of radiation source(s) in that location spanning the region of the dashed lines and between the dashed lines. Although FIG. 2A illustrates an embodiment in which substrate 104 is a single opaque strip, any of the other embodiments of radiation applicator 100 may be used instead.

If substrate 104 is transparent or translucent to the radiation source(s) 102, then substrate 104 could be placed between radiation source(s) 102 and lesion 202. An advantage to placing substrate 104 between radiation source(s) 102 and lesion 202 is that radiation source(s) 102 may be left exposed to air, which may facilitate passive and/or active (e.g. a thermoelectric cooling device) cooling of radiation source(s) 102. Additional structural elements such as fins or other heat diffusing, heat dispersing, and/or heat sinking elements can be attached or manufactured on substrate 104; additionally, electrodes or other conductive paths can be applied to or manufactured on substrate 104. Processes such as chemical or vapor deposition processes can be used to deposit heat conducting or electrically conducting materials on substrate 104. Alternatively, the radiation source(s) 102 may be adapted to traverse the material so that the light emitting face is placed between the substrate 104 and lesion 202 and the electrical connections and heat generating components are such that they direct heat away from the lesion 202 (and/or electricity toward the radiation source(s) 102) through the substrate 104, and then to the ambient atmosphere. Also, substrate 104 may include elements and/or structural features that facilitate uniform irradiation of lesion 202, such as by scattering or focusing the radiation emitted from radiation source(s) 102. One example of a scattering structure is a substrate having one or both of its outer surface and its surface facing radiation source(s) 102 roughened or textured. Another example of a scattering structure is a substrate having particles (e.g. titanium oxide and/or aluminum oxide) embedded within it that have a different index of refraction than the substrate. Any one of, any combination of, or all of these scattering structures may be included in substrate 104 (and/or within other layers) for uniformly irradiating lesion 202.

An advantage in placing radiation source(s) 102 between substrate 104 and lesion 202 is that a greater percentage of the radiation generated is incident upon lesion 202. Consequently, the power efficiency may be greater without substrate 104 intervening between radiation source(s) 102 and lesion 202 than with substrate 104 in an intervening position.

FIG. 2B illustrates a target body surface, such as a layer of skin 270. The layer of skin is comprised of the stratum corneum 250, the stratum lucidum 252, the stratum granulosum 254, the germitive layer 256, 258 and the dermis 260. Lesion 202 is depicted crossing all of the layers for purposes of illustration. However, as will be appreciated by those skilled in the art, the layers of the skin affected by the lesion will be determined by the type and extent of medical condition associated with the skin, e.g. psoriasis, contact dermatitis, vitiligo, acne, atopic dermatitis, cellulite, collagen laxity associated with aging, and skin cancer. In this illustration, the radiation applicator 100 is positioned on the target body surface to be treated such that the radiation source(s) 102 will be in proximity to the lesion 202. As described above and below, the radiation applicator can contain a multitude of radiation generators which alone or in combination can apply radiation to difference depths within the lesion. For example, infrared wavelengths can be used to penetrate the deeper parts of the lesion whereas ultraviolet wavelengths can be used to penetrate the more superficial portions of the lesion. Photosensitizers can further be utilized to modulate the depth of penetration. For example, if a red light absorbing photosensitizer is applied superficially to the lesion, then the superficial portion of the lesion is treated with the red light. In this embodiment, the depth wherein light activates the photosensitizer is determined by the depth where the photosensitizer is placed or level it is absorbed to. If the photosensitizer is injected 2 mm underneath the skin, then the light will be absorbed in this layer assuming that light is not absorbed in the more superficial layers of the skin.

FIG. 3 shows a block diagram of an example of radiation applicator 100. Similar to FIG. 1, FIG. 3 shows radiation source(s) 102 (e.g. 102a-102n), substrate 104, and region 106. Additionally, FIG. 3 shows controller 302, power source 304, and electrical connectors 306. In other embodiments, radiation applicator 100 may not have all of the components associated with FIG. 3 and/or may have other components in addition to, or instead of, those depicted for purposes of illustration with FIG. 3.

Radiation source(s) 102, substrate 104, and region 106 were described in conjunction with FIGS. 1 and 2A-B. Controller 302 may include a processor and/or a specialized circuit for controlling radiation source(s) 102. Controller 302 may be a microcontroller. For example, controller 302 may have a width and/or length that are less than 5 cm, less than 4 cm, less than 3 cm, less than 2 cm, or less than 1 cm. As discussed above, controller 302 can be adapted and configured to control radiation source(s) 102 and may control how long and/or which ones of radiation source(s) 102 is/are powered on. Additionally, or alternatively, controller 302 may control the wavelength, frequency, and/or the intensity of the radiation of radiation source(s) 102. In addition, controller 302 can integrate feedback from reflectance sensors (not shown) associated with the device 100 which relay real-time information about the state of the lesion or of the surrounding skin. Controller 302 further has the ability to be programmed from a device (e.g. a wireless or wired device such as a computer, personal digital assistant, etc.) outside the radiation applicator 100.

In an embodiment, controller 302 may relieve the patient and/or doctor from the task of keeping track of the time that the therapy has been applied. For example, controller 302 may track the total amount of time that each individual one of radiation source(s) 102 and/or each of a plurality of groups of radiations source(s) 102 has been in use. In other words, each of radiation source(s) 102 may be turned on and off in cycles, and controller 302 or a timer (not shown) may keep track of the total amount of time and/or total energy that any given radiation source(s) has been kept on. The controller in some embodiments facilitates the portability of the device. If the dosage being applied to the patient is not being monitored by the physician or the patient it would therefore be possible that too high a dose is delivered to the treatment area. With a controller various groups of radiation source(s) 102 may be turned on and off together, separately or not at all while keeping track of how long an individual radiation source has been on and/or how long a group of radiation source(s) associated with this individual radiation source has been on, (because the group of radiation source(s) and any individual radiation source within the group is expected to have been on for the same amount of time). In some embodiments, the patch is provided with a computer interface so that the patient or doctor programs the computer interface and subsequently the patch to achieve a specific dose on one or more target areas. For example, the user of the computer interface determines the region to be treated and the dosage to be applied. This methodology ensures that a specific dosage is applied to a specific (e.g. diseased) location on the body surface. In this way, the ideal toxicity: efficacy ratio can be obtained.

When a particular one of, or group of, radiation source(s) 102, has delivered a predetermined therapeutic dose of energy, radiation controller 302 turns off or otherwise decreases its applied dose 102. A therapeutic dose of radiation may be an amount of radiation that has been determined to be the maximum or slightly less than the maximum tolerable dose during a particular treatment session. Tolerable can mean a sunburn in the case of ultraviolet light applied to the skin. Alternatively, a therapeutic dose of radiation may be an amount of radiation that has been determined to be appropriate for a particular disorder or a particular treatment session. As will be appreciated by those skilled in the art, different disorders may have different therapeutic doses. For example, a therapeutic dose may be a sub-threshold Minimal Erythemal Dose (MED) in some skin disorders. As another example, a therapeutic dose may be reached when all the radiation source(s) 102 or when all of the groups of radiation source(s) 102 have delivered 100-600 mJ/cm² (of ultraviolet light in the 295-320 nm range for example) to body portion 200. Consequently, when all of the groups of radiation source(s) 102 have delivered 100-600 mJ/cm² to portion 200, the therapy for that region is finished.

A method of applying radiation therapy in the context of this invention includes the steps of: visualizing a body surface to be treated; mapping the body surface to be treated in a device interface; delineating an area of the body surface to apply radiation therapy to; programming a topologic dosage map to the radiation therapy device via the computer interface; applying the radiation therapy device to the body surface in an orientation where the topologic dosage map align with the underlying disease being treated; and allowing the radiation therapy device to function autonomously after the device applied to the body surface.

In some embodiments, doses are applied to the treatment region on a continuous basis and the maximum therapeutic dose guides the therapy. For example, a time can be defined, over which a maximal dose cannot be exceeded. Using the skin as an example, an MED, a fraction of an MED, or a multiple of an MED can be given to a body region over a 30 second period, a 12 hour period, a 24 hour period, a 48 hour period, or over any period of time in between or other time chosen by the patient or the physician; it is also conceivable that erythema (in the skin for example) can be avoided altogether when the dose is given over a long period of time. After this period of time, another dose is give to the same region or another region. In other embodiments, the dose delivered to the region with the lesion can exceed the toxicity dose of the non-lesional region because the radiation device can selectively apply radiation to one region versus another region and the application region can be programmed into the device by the physician or the patient. For example, in the case of psoriasis, the dose that can be delivered to the region with a psoriatic plaque can exceed the minimal erythemal dose by a factor of, for example, 2,3,4,5,6,7,8,9, or 10 because the psoriatic region is more resistant to radiation than normal skin. With most existing devices, it is not possible to define a treatment region while avoiding non-treatment regions. It is typically the responsibility of the operator of the device to apply radiation to unhealthy regions and not healthy regions.

In an embodiment, radiation applicator 100 maybe programmed by the patient or by the physician to deliver a particular therapy over a period of time. In an embodiment, controller 302 may be programmed to calibrate radiation applicator 100 or have a calibration mode during which radiation applicator 100 is calibrated. For example, radiation applicator 100 may be calibrated for the patient prior to applying a therapy (e.g. due to the fact that different patients have different sensitivities to light due to differing amounts of melanin contained in a patient's skin).

During calibration, radiation applicator 100 is placed on a portion of the body that is unaffected by the disorder that portion 202 is affected by. For example, radiation applicator 100 is placed on a portion of healthy skin typically unexposed to sunlight (e.g., the gluteal region). Next, escalating doses of radiation are applied to the skin. The dose, which after 24 hours produces a superficial redness of the skin from dilation of the capillaries, or erythema, is called the Minimal Erythemal Dose (MED). Controller 302 may be programmed to automatically apply the escalating doses to different regions under radiation applicator 100. After 24 hours, the MED is determined by the region which has a perceptible erythema, or redness. The patient's MED is then programmed into controller 302 and the MED, or an amount of radiation slightly less than the MED, becomes the calibrating dose for the particular patient. This device configuration can also be utilized to diagnose disease. For example, the disease state, polymorphic light eruption, is a disease in which an allergic response occurs with light exposure. It is typically a tedious process to diagnose the specific wavelengths and/or power required for the allergic response to light, requiring a large amount of technician time and equipment. A radiation device 100 can be used for diagnosis in some embodiments. For example, radiation device 100 can have a multitude of radiation sources with different wavelengths, each of which deliver specific energies in different wavelength bands. The radiation device can then be applied to a body surface (e.g. skin) with a program to deliver a specific wavelength and/or dose to different body surface areas under the device over specific times. After the doses are delivered, the region which develops the skin reaction can be determined by observing the region which has the reaction. Similarly, a radiation device can be used to determine body reactions to photosensitizing pharmaceuticals, cosmetics, natriceuticals, and sunblocks. In the case of sunblocking compounds, various compounds can be placed underneath the radiation device and prescribed doses of radiation programmed into the patch. The radiation applicator in these diagnostic embodiments can further be adapted to fit animals, such as pigs, rats or mice which are often used to test the potential photosensitizing compounds.

To treat a disease such as psoriasis, doses are typically related to the MED. For example, a standard course of therapy consists of 3 weeks of treatments, 3 times per week, with each treatment consisting of 1-3 MED depending on what the patient can tolerate. It is difficult, if not impossible, for the treatment area to be well-controlled; some areas of non-diseased skin will receive treatment. It is these areas which limit the amount of radiation which the affected areas can receive. Further, the risk of skin cancer is increased in the areas unaffected by disease but which are nonetheless exposed to radiation because of the non-specificity of the radiation applicator. Furthermore, the treatments are given three times per week solely because the unaffected skin must heal before the next treatment. A device which could limit treatment area to the lesional area could be beneficial in that the treatment dose and/or frequency could be increased and the total treatment time decreased. Furthermore, a device which does not require the patient to be at the physician's office or otherwise schedule time for a treatment could be highly beneficial in many patients and result in greater treatment protocol compliance by the patient which in turn would lead to greater efficacy of patient treatment. With radiation applicator 100, the treatment region can be finely tuned by the patient and/or physician. In embodiments where the device is worn by the patient, the patients do not have to stop what they are doing (e.g. work, sleep, exercise, etc.) to receive treatments.

In embodiments in which controller 302 is kept small (e.g., in embodiments in which controller 302 is a microcontroller), the small size facilitates making radiation applicator 100 portable. Controller 304 may be located on substrate 104. In an embodiment, controller 302 is an integral part of substrate 104 (e.g., controller 302 may be embedded within substrate 104). Controller 302 switches power between different radiation source(s) 102, so that some of radiation source(s) 102 are powered on while others are powered off. In an embodiment, controller 302 may never, or only infrequently, power on all of radiations sources 102 simultaneously. Alternatively, controller 302 will have at least some period of time when not all of radiation source(s) 102 are powered on simultaneously. If controller 302 does not keep at least some of radiation source(s) 102 (although not necessarily the same radiation source(s) 102) off all of the time, nearly all of the time, most of the time, or at least some of the time, the current required for operation may be very high and may generate excess heat in addition to requiring a very large power source as compared to the operating current required, the heat generated, and the size of the power source when some or all radiation source(s) 102 are turned on and off to conserve power. A large power source and excessive heat dissipation requirements may require component sizes that limit the portability of a radiation applicator and the ease and/or comfort with which radiation applicator can be worn. The selective activation of radiation source(s) 102 and the duration of radiation source activation time (e.g., the duty cycle) may be based upon the power capacity of a power source, which is kept small enough to keep radiation applicator portable and self-contained. Alternatively, or in addition to, the amount of time that a given one of radiation source(s) 102 is kept on may be based upon cooling considerations and/or a desired intensity of radiation that is expected to be therapeutic. In an alternative embodiment, radiation applicator 100 is connected to an external computer or an external controller during, before, or after operation or is at least in part controlled wirelessly by a remote unit during, before, or after treatment. Additionally, as will be appreciated, the power source may be contained in a water-resistant, or water-proof housing (not shown). The housing may be configured to be connectable to the radiation applicator 100 in such a manner that the connectors between the radiation applicator 100 and the housing can be connected in a manner that provides a secure moisture resistant connection.

Using a microcontroller for controller 302 may simplify the structure of the radiation applicator as well. For example, in an embodiment in which each of radiation source(s) (e.g. 102a) is on for only a short period of time before being turned off and another one of radiations sources (102n) being turned on, heat transfer through substrate 104 is not as large an issue as it would be if all of radiation source(s) 102 (e.g. 102a-102n) were run continuously. Consequently, there may not be any need to pump a fluid through radiation applicator 100 for cooling. Similarly, there may not be any need for perforating substrate 104 for cooling.

Optionally, radiation applicator 100 may include one or more detectors to detect whether the body surface of the patient has been harmed and/or may be harmed soon. For example, radiation applicator 100 may include one or more detectors to detect erythema. The detectors may detect erythema by detecting the color of a target portion of the body or a change in the color of a target portion of the body (e.g., skin color). In another embodiment, there may be detectors for detecting the color, moisture, and/or temperature of the target portion being irradiated to ensure that the portion irradiated is not being damaged by the radiation. Optionally, after detecting erythema and/or any other condition indicative that radiation applicator 100 may have harmed, or may harm, the target portion being irradiated, controller 302 may automatically turn off radiation source(s) 102 (e.g. 102a-102n). Controller 302 may turn off the radiation source(s) 102 associated with the erythemal region as part of the calibration routine and/or as a safety feature during a treatment in response to input from one or more detectors concerning the condition of the region being irradiated (e.g., after an erythemal condition is detected).

Although FIG. 3 shows an example in which there is only one controller 302, there may be a plurality of controllers. Each one of radiation of sources 102 or each group of radiation source(s) 102 may have its own controller. There may be a system of controllers in which there is one master controller that controls other local controllers, and the local controllers may control individual ones of and/or groups of radiation source(s) 102. Optionally, controller 302 may have one or more input ports or input devices that may be used for programming, inputting parameters, and/or setting controller 302 according to a particular therapy, which may be based on a calibration that was performed. The programming, input parameters, and/or settings may be entered by a patient, entered by a doctor, and/or automatically entered as part of a calibration and/or setup procedure. Examples of inputs include, but are not limited to, Bluetooth®, USB, optical, or any other wired or wireless connections.

Power source 304 powers controller 302 and/or radiation source(s) 102 are provided for as shown in FIG. 3. In the example of FIG. 3, power source 304 supplies power to radiation source(s) 102 via controller 302. Power source 304 may be one or more batteries, a power supply that plugs into an outlet, and/or one or more photocells for recharging one or more batteries. Power source 304 may include one or more flat, disc-shaped batteries, which may be less than 2 or 3 millimeters thick, and less than 1 or 2 centimeters in diameter. For example, power source 304 may be one or more lithium ion batteries. Alternatively, power source 304 may be one or more nickel cadmium, AA, and/or AAA batteries, for example. Although in the example of FIG. 3 there is only one power source shown, there may be a plurality of power sources located in a plurality of locations within radiation applicator 100. Each one of, or each group of, radiation source(s) 102 (e.g. 102a-102n) may have their own power source. Power source 304 may be located on substrate 104. In an embodiment, power source 304 is an integral part of substrate 104 (e.g., power source 304 may be embedded within substrate 104). In another embodiment, power source 304 is one or more photovoltaic cells.

Depending upon the configuration of the radiation applicator 100, the weight of the device can range from, for example, 0.5 g to 200 g, more preferably from 0.5 g to 100 g, and even more preferably from 0.5 g to 10 g. As will be appreciated by those skilled in the art, these weight ranges are meant to be illustrative of a reasonable weight which an individual can tolerate. Other weight ranges could be used without departing from the scope of the invention.

Electrical connections 306 communicatively connect radiation source(s) 102 (e.g. 102a-102n) to controller 302 so that controller 302 is capable of controlling radiation source(s) 102. Electrical connections 306 also electrically connect power source 304 to radiation source(s) 102, via controller 302, such that power source 304 supplies power to radiation source(s) 102. Electrical connections 306 may include a bus that sends signals to individual radiation source(s) 102. Alternatively, electrical connections 306 may include individual pairs of electrical connections, where each pair links one of, or one group of, radiation source(s) 102 directly to controller 302.

Electrical connections 306 may be attached to substrate 104 individually or they may be created directly on the material by a process of photolithography, electrodeposition, chemical vapor deposition, and/or physical vapor deposition. Alternatively, electrical connections 306 are embedded in a flexible insulating film, the entire film then being attached to substrate 104. Electrical connections 306 can be wire-bonded connections produced using a wire bonding process well-known in the LED arts. These connections are three dimensional and can be protected via material film around the connections. One representative example of a flexible film is a silicone film. A silicone film can be used to embed wires which lead to a connector such as a computer pin connector. After the bus and the wires are embedded, the film can be mated with another film which is a radiative device 100 or a heat conducting film. When the two sides (film with the wires and film with the LEDs) are mated to one another, the device is electrically connected.

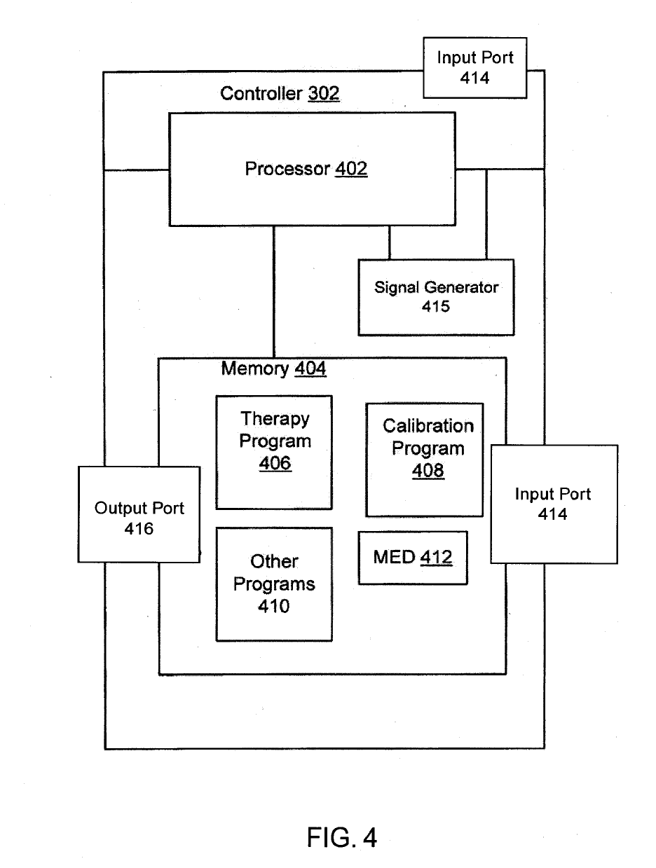

FIG. 4 shows a block diagram of an example of controller 302. Controller 302 may include processor 402, memory 404, and signal generator 415. Memory 404 may have a therapy program 406, calibration program 408, and/or other programs 410. Memory 404 may store MED 412 and/or other parameters such as the dose history previously applied to the patient. Controller 302 may also include one or more input ports 414 and one or more output ports 416. In other embodiments, controller 302 may not have all of the components associated with FIG. 4 and/or may have other components in addition to or instead of those associated with FIG. 4.

Processor 402 performs the therapy program and/or calibration programs referred to above and/or other programs. Memory 404 may include one or more machine-readable mediums that may store a variety of different types of information.

The term machine-readable medium is used to refer to any medium capable of carrying information that is readable by a machine, such as processor 402. One example of a machine-readable medium is a computer-readable medium. Although machine-readable medium of memory 404 is capable of storing information for a period of time that is longer than the time required for transferring information through memory 404, the term machine-readable medium may also include mediums that carry information while the information that is in transit from one location to another, such as copper wire and/or optical fiber.

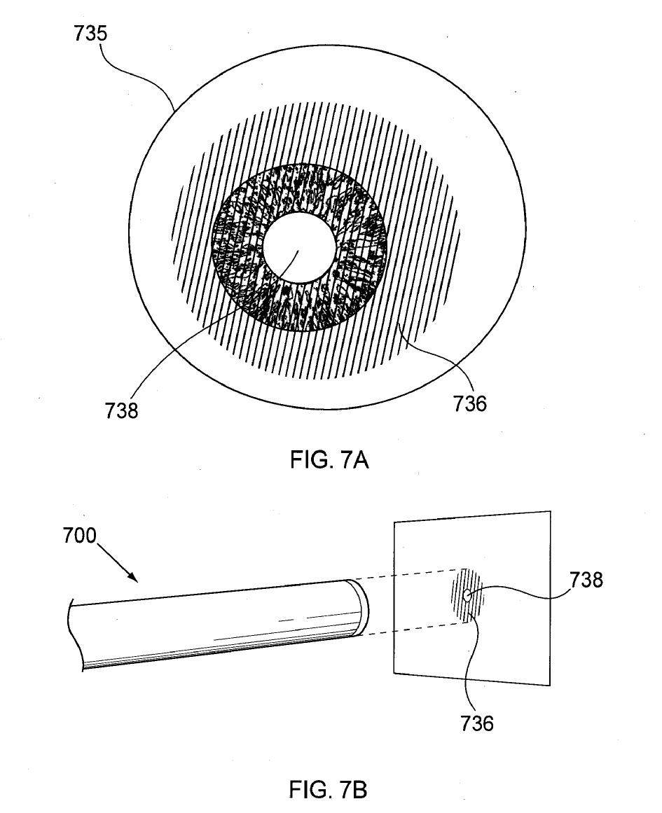

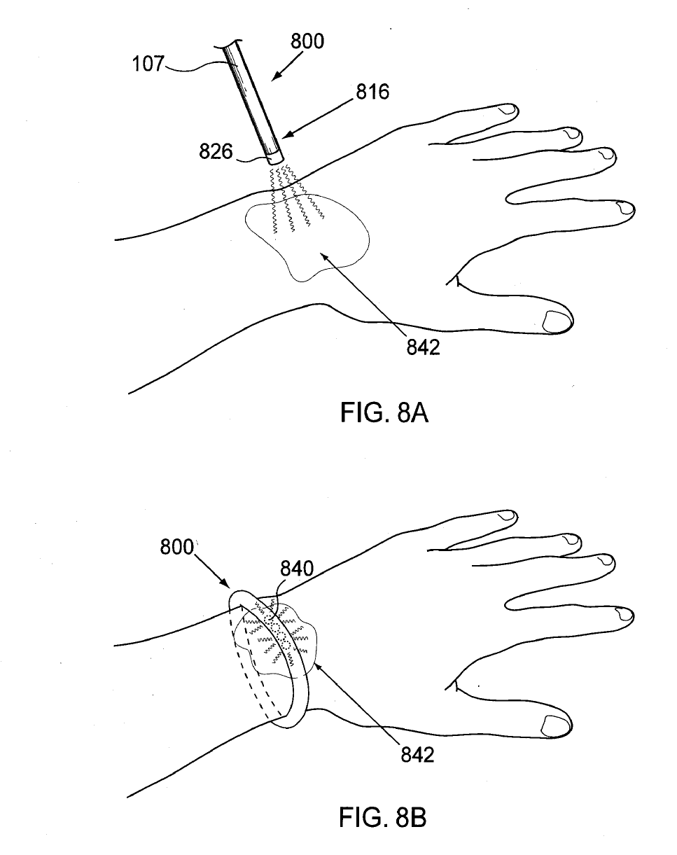

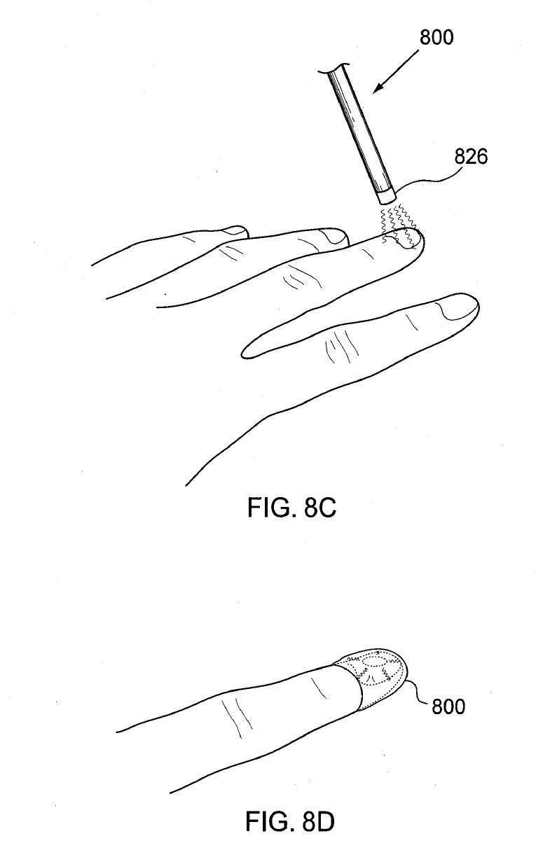

Memory 404 stores programs that are executed by processor 402 and/or parameters used by those programs. In this specification, the word program is used to refer to any group of one or more instructions that cause a processor to perform at least part of a task when the one or more instructions are executed. In the example of FIG. 4, memory 404 may store therapy program 406 and/or calibration program 408 and or dose history program. Therapy program 406 and calibration program 408 include one or more instructions that cause processor 402 to perform the therapy and the calibration discussed in conjunction with FIGS. 1-3, respectively. Memory 404 may also store other programs 410, which are optional. If present, other programs 410 may include one or more other programs entered by the doctor or patient. Therapy program 406 will be discussed further in conjunction with FIG. 8, and calibration program 408 will be discussed further in conjunction with FIG. 7.

MED 412 (such as discussed in conjunction with FIG. 3) and/or other parameters may be entered by a patient or doctor and/or may be determined and/or stored automatically. One or more input ports 414 may be connected to one or more input devices for entering programs and/or parameters into memory 404. One or more input ports 414 may also receive input from one or more detectors used for calibrating radiation applicator 100. One or more input ports 414 may be useable as an interface to a computer or other machine that is used for programming controller 302. One or more input ports 414 may be useable for downloading programs, an MED, configuration parameters, and/or other information to controller 302. Input ports 414 may include an input port for a wireless signal (e.g., an antenna). Alternatively, a computer or other machine may be attached to one or more input ports 414, and used to either directly control radiation sources 102 or control radiation source(s) 102 via controller 302.

Signal generator 415 may produce a variety of different signals that vary in pulse width, pulse height, and/or pulse shape. Signal generator 415 may produce signals having different duty cycles based on the capabilities of power source 304, and based on how much heat is generated by radiation source(s) 102 (e.g. 102a-102n) while in an on state and/or a desired therapy. Signal generator 415 may be controlled by processor 402. Signal generator 415 is optional. In an embodiment in which signal generator 415 is not present, processor 402 may address radiation source(s) 102 directly.

One or more output ports 416 may be associated with the controller 302 and may be connected, via electrical connections 306, to radiation source(s) 102. There may be one output port 416 for each one of, or each group of, radiation source(s) 102. One or more output ports 416 may be capable of being connected to one or more output devices, such as a monitor and/or display. By connecting an output device, it may be possible to view programs and/or parameters entered into memory 404 to aid in programming processor 402 and/or debugging one of the programs stored on memory 404. If signal generator 415 is present, some of the one or more output ports 416 may be connected to corresponding outputs of signal generator 415, and some of the one or more output ports 406 may be connected directly to processor 402 for communicating with an external device, such as a computer or terminal.

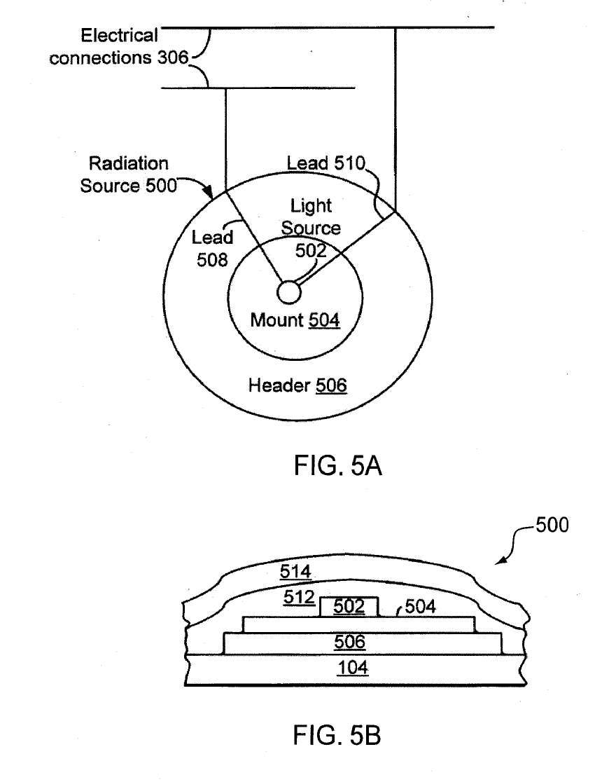

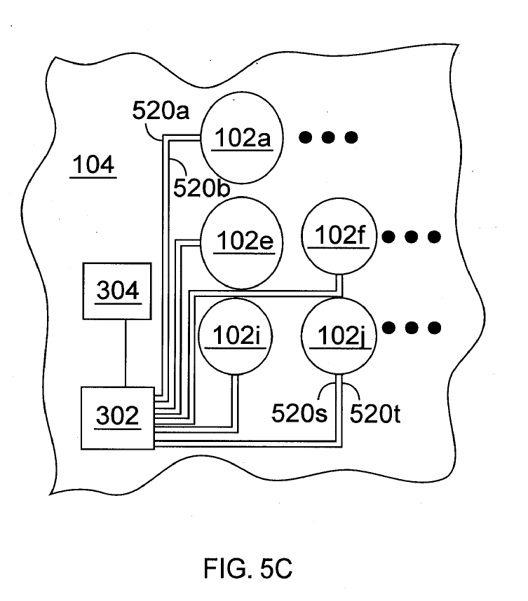

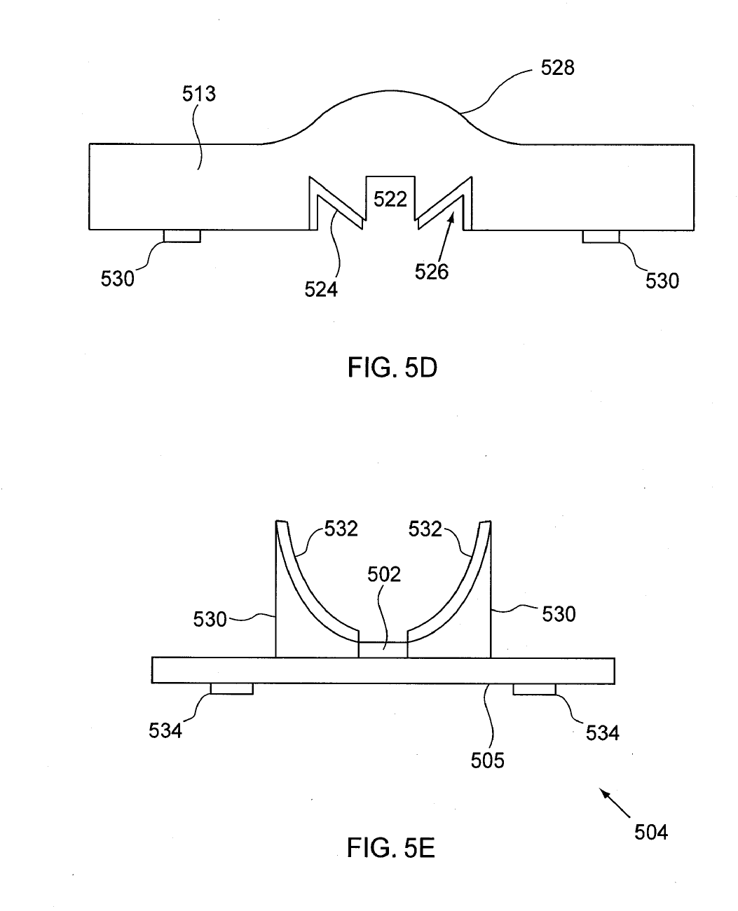

FIG. 5A shows a schematic diagram of an example of radiation source 500. Radiation source 500 may include the actual radiation source 502, such as a light source, and its supporting elements which allow the radiation source to function. For example, if the radiation source is a light emitting diode (LED), the supporting elements can include mount 504, header 506, lead 508, and lead 510; these supporting elements can be referred to as the radiation source module. In other embodiments, radiation source (or radiation source module) 500 may not have all of the components associated with FIG. 5A and/or may have other components in addition to, or instead of, those associated with FIG. 5A. Furthermore, as would be recognized by those skilled in the art, many variations of these basic components are possible. For example, the mount 504 could be made from any of many shapes, sizes, thicknesses, or from materials such as Berylium Oxide (BeO), Aluminum Nitride (AlN), alumina, aluminum, copper, steel, MgF, or a semiconductor (e.g. silicon). The leads 508, 510 can be made from copper, silver, gold, alloys, or polymers as would be recognized by those skilled in the art. Header 506 can be made from a variety of materials or made into many shapes. Header 506 can also contain features necessary for heat transfer such as fins or dimples to increase the surface area of the header. The header can also be manufactured by depositing or molding metal (e.g. Kovar®, an alloy of iron, nickel and/or cobalt which has similar thermal expansion properties to glass, Westinghouse Electric & Manufacturing, Pittsburgh Pa.) directly onto a flexible material (e.g. silicone), which is part of the applicator 104 in FIG. 1. The radiation source can then be placed, using a die bonder, onto the deposited Kovar, after which wire bonds or soldered welds can be used to attach the radiation sources to a power circuit. Alternatively, the wire bonds can also be deposited on the flexible substrate (e.g. surface 104 in FIG. 1) using deposition processes such as electrodeposition, chemical vapor deposition, or physical vapor deposition.

Radiation source 502 may be a surface mount LED, or LED die, such as a UV LED die, blue light LED die, white light surface mount (SMD), Infrared (IR) LED or SMD, or UV LED SMD. As another example, radiation source 502 may be a small light bulb, resistive heater, or a device for generating microwaves, radiofrequency energy, x-rays, and/or radio frequency light. More specifically, radiation source 502 can emit energy in the immunosuppressive or anti-infective range of the ultraviolet spectrum. Wavelengths included in the immunosuppressive range of the ultraviolet spectrum include those from 295 nm to 320 nm and/or from 340 nm to 400 nm. In other embodiments where it is desired to treat infectious agents, radiation source 502 can emit ultraviolet light in the range 250-300 nm.

In an embodiment where radiation source 502 is a light source, mount 504 may hold light source 502 in place. Mount 504 may include a heat sink, circuit board, or a circuit board on top of a heat sink (e.g., a passive heat sink to diffuse heat over a larger surface area or an active sink to electrically pump heat away from the light generating regions). One example of a circuit board (sub-mount) is a gold-patterned ceramic such as beryllium-oxide (BeO) or aluminum nitride (AlN); the ceramic can act as a heat sink or a highly conducting heat transfer element through which heat conducts to the heat sink. Mount 504 may be a material such as Kovar alloy, which can act as a heat sink in addition to the ceramic material and is a very good material to bond beryllium oxide or aluminum nitride to because it (Kovar alloy) has a very similar coefficient of heat expansion. If mount 504 includes a heat sink, mount 504 may reduce the likelihood of light source 502 overheating and/or may otherwise extend the lifetime of light source 502 so that light source 502 lasts longer with a higher optical output per electrical input (efficiency) than if there were there no heat sink. Although in the example of FIG. 5, there is only one light source 502 on mount 504, there may be plurality of light sources on each mount 504. Light source 502 (e.g., an individual or multitude of UV LEDs) may be attached (e.g., bonded) to mount 504 using a eutectic metal or a solder such as gold-tin, lead-tin, other applicable eutectic solder material. Optionally, mount 504 may be textured (e.g., roughened) for scattering light or polished for specularly reflecting light. Mount 504 may be shaped for concentrating, diffusing, collimating, or dispersing light from light sources 502. Mount 504 may be flat, concave, or convex. If mount 504 is concave or convex, mount 504 may be elliptical, spherical, or hyperbolic, for example. Mount 504 may be composed of, or coated with, a reflecting metal such as aluminum or aluminum derivative. Mount 504 can additionally contain three-dimensional features 530 which are deposited on mount 504 (FIG. 5E).

Further, with respect to FIG. 5E a radiation source is depicted in the center of two three-dimensional pillars 534. The pillars can be deposited onto mount 505 or they can be attached after being made by another means. Typical attachment processes can include a press fit, eutectic mount, adhesive mounting, ultrasonic welding, and light based curing. Mounting elements 530 can be electrical mounts, a material solely intended for the mounting process, a material to facilitate heat transfer, or a combination thereof. The radiation source (e.g. a light source) can be placed in between the three-dimensional pillars 534 so that the radiation will reflect or refract forward from the three-dimensional pillars 534 in a pre-determined pattern outward to the body surface. As will be appreciate by those of skill in the art, the three-dimensional pillars 534 can assume any of a variety of configurations other than the pillars depicted without departing from the scope of the invention.

An advantage of placing pillars 534 around the radiation source or multiple individual radiation sources is that the radiation from the individual radiation sources can be captured independently from other radiation sources nearby. Such an arrangement can optimize light extraction and can direct the radiation in specific directions. Three-dimensional pillars 534 can be deposited on the surface 505 of the mount using processes such as eletrodeposition, chemical vapor deposition, physical vapor deposition, micromolding, electroforming, or other deposition processes known to those skilled in the art. In one example, mount 504 is made from a ceramic such as Berylium Oxide or Aluminum Nitride. Standard physical vapor deposition processes can be used to then deposit conducting metallic layers such as gold or a eutectic metal such as gold-tin on the ceramic. With a conducting surface such as gold deposited on the ceramic, additional features can then be deposited (e.g. with an electrodeposition process) on the conducting metal which would reflect, focus, concentrate, disperse, or otherwise condition light. In another example, three dimensional features are not deposited directly but are produced in separate molds which are then applied to the surface SOS of the mount 504. When the surface pattern in the mount 504 is made from a eutectic metal, the mold placed on the mount surface and heat is then applied to the mount 504. The heat can weld the eutectic metal to the three-dimensional piece in the mold; after cooling, the mold is removed, leaving the mount 504 with a three-dimensional feature 530 welded to it. A combination of these processes can also be used in which three-dimensional features 534 are fabricated and then additional layers 532 are deposited on top of the three-dimensional features. For example, UV reflecting aluminum could be deposited on top of the three-dimensional features 534 on the mount 504. Light is then directed from radiation source 502 using one or all of these processes and/or structures.

Header 506 may protect light source 502 and mount 504 from being separated. Although in the example of FIG. 5A-E, header 506 has only one mount 504, there may be plurality of mounts 504 and each mount may have only one light source or may have a plurality of light sources. Similar to mount 504, header 506 may be shaped for concentrating, diffusing, collimating, dispersing, or otherwise reflecting light (e.g., with an aluminum reflecting layer) light from light sources 502. Header 506 may be flat, concave, or convex. If header 506 is concave or convex, header 506 may be elliptical, spherical, or hyperbolic, for example. Alternatively, there may be another optical component in addition to, or instead of, shaping and/or texturing mount 504 and/or packaging header 506 to have particular optical properties. Specifically, this additional optical component may be shaped for concentrating, diffusing, collimating, or dispersing light from light sources 502. The additional optical component may be flat, concave (for dispersing the radiation), or convex for concentrating the radiation. If the additional optical component is concave or convex, the additional optical component may be elliptical, spherical, or hyperbolic, for example. Header 506 can also contain three-dimensional microfabricated components as described above in the mount. The same or similar processes can be employed for the header.

In an embodiment, mount 504 and header 506 are separate components that are attached to one another. In another embodiment, mount 504 and header 506 may be two parts of the same component and/or only one of mount 504 and header 506 are used. If there is more than one light source on each mount 504 and/or within each header 506, the light sources may all have the same spectrum and/or may be associated with the same peak wavelength. Alternatively, there may be different light sources having different spectrums and/or peak wavelengths that are located on the same mount 504 and/or one the same header 506.

The leads 508, 510 supply power to light source 502 for activating light source 502 and keeping light source 502 lit. Further, leads 508, 510 may be connected to larger leads on substrate 104 that bring electricity to radiation source 502 (e.g., leads 508 and 510 may be connected to electrical connections 306). As will be appreciated by those skilled in the art, leads 510, 508 may be made from an alloyed, eutectic or non-alloyed, metal placed on or bonded to mount 504. Thus, current from power source 304 flows to controller 302, through electrical connections 306, and to one or more of radiation source(s) 102 (e.g., to leads 508 and 510, and then to light source 502, such as an UV LED), resulting in light, such as UV light, being output and subsequently biologic effect.

FIG. 5B shows a cross-section of an embodiment of radiation applicator 100. The embodiment of FIG. 5B includes flexible substrate 104, light source 502, mount 504, header 506, spectral conditioner 512, and optional patient interface 514. In other embodiments, radiation applicator 100 may not have all of the components associated with FIG. 5B and/or may have other components in addition to, or instead of, those associated with FIG. 5B.

Substrate 104 is discussed above in conjunction with FIG. 1 and elsewhere. Light source 502, mount 504, and header 506 are discussed above in conjunction with FIG. 5. Spectral conditioner 512 covers and may protect light source 502 from damage and/or may condition the radiation in one or more ways before it reaches the lesion. Spectral conditioner 512 may be one continuous layer of material that extends over all of region 106 or over all of substrate 104. Alternatively, spectral conditioner 512 may be a collection of patches of material, where each patch conditions the radiation from at least one light source, such as light source 502. In this embodiment, when the spectral conditioner 512 is a patch and individually covers one light source, the entire light source, including the covering 513, header 506, and mount 504 can be individually removed from the material 104 and then replaced on material 104. Depending on the embodiment, spectral conditioner 512 may cover a larger area than light source 502 but smaller than or equal to mount 504, cover a larger area than mount 504 but smaller than or equal to header 506, or cover a larger area than header 506 but not large enough to reach a covering of an adjacent radiation source.

Spectral conditioner 512 may make radiation applicator 100 more comfortable to wear, because the surface of spectral conditioner 512 that contacts the body portion can be smoother than the surface of radiation applicator 100 than if spectral conditioner 512 were not present. Spectral conditioner 512 and substrate 104 may form two layers of material, with light sources 102 sandwiched in between. Spectral conditioner 512 may be a layer of material, which may be transparent or translucent (e.g. to ultraviolet light between 250 nm and 320 nm), while a substrate 104 may be transparent, opaque, translucent, or reflective. If substrate 104 is reflective, substrate 104 may be specularly reflective or may scatter light. By making substrate 104 reflective, the efficiency of radiation applicator 100 is improved as compared to where substrate 104 is not reflective. By making either or both of substrate 104 and covering 513 a light scattering material, the uniformity of the irradiation may be improved as compared to if substrate 104 and/or spectral conditioner 512 do not scatter light. Spectral conditioner 512 may be made to scatter light using any of the structures discussed above in conjunction with the discussion of substrate 104 of FIG. 2. Spectral conditioner 512 may reduce efficiency (depending upon how much radiation it absorbs or otherwise prevents for reaching the patient), but may improve the uniformity of the irradiation and/or comfort to the patient.

Optional patient interface 514 may be an adhesive to help radiation applicator 100 adhere to the body portion being treated. Optional patient interface 514 may be a layer of adhesive material (e.g., glue) that partially or completely covers one surface of radiation applicator 100, such as covering 513. Optional adhesive 514 may be included in an embodiment in which radiation applicator 100 is a bandage that sticks to a portion of skin of a patient, for example. Optional adhesive 514 may the adhesive discussed in conjunction with FIG. 1 and/or substrate 104. In addition to glue, patient interface 514 may incorporate therapeutic substances designed to prevent damage and/or enhance the therapeutic efficacy of the radiation delivered by radiation applicator 100. Examples of potentially protective compounds include titanium oxide, zinc oxide, and others well-known to those skilled in the art. Examples of compounds to improve efficacy can include photosensitizers such as the broad categories of psoralens, the porphyrin family, and other photosensitizers which are well-known in the art. FIG. 5C shows a bock diagram of an example of an embodiment of radiation applicator 100. FIG. 5C includes radiations sources 102a, 102b, 102e, 102f, 102i, and 102j, substrate 104, controller 302, power source 304, and electrical connections 520 (such as 520a-520t). In other embodiments, radiation applicator 100 may not have all of the components associated with FIG. 5C and/or may have other components in addition to or instead of those associated with FIG. 5C.

Radiation source(s) 102a, 102e, 102f, 102i, and 102j are specific ones of, or specific groups of, radiation source(s) 102 (e.g. 102a-102n), which are discussed in conjunction with FIGS. 1 and 5A shown in FIG. 5C. The sets of three dots after radiation source(s) 102a, 102f, and 102j represent any number of radiations sources. Although pairs of letters, such as “e” and “f,” and “i” and “j,” may represent pairs of consecutive numbers that are smaller than the number represented by “n,” there may be any number of radiation source(s) between radiation source(s) 102b and 102e, between radiation source(s) 102f and 102i, and between radiation source(s) 102j and 102n. Substrate 104 is discussed in conjunction with FIGS. 1 and 5B and elsewhere. Controller 302 and power source 304 are discussed in conjunction with FIG. 3 and elsewhere.

Electrical connections 520 (e.g. 520a-520t) are paired with one another. Each pair completes a circuit between controller 302 and one of radiation source(s) 102 (e.g. 102a-102n). The pattern of electrical connections 520a-520n is different than electrical connections 306 (FIG. 3). In this embodiment, each radiation source or group of radiation source(s) has its own ground or return electrode and can be controlled independently by controller 302.

Turning now to FIG. 5D, a close-up of a molded covering 513 with optical components built-in is depicted. In this embodiment, covering 513 is placed over the radiation source which then resides in space 522. The covering 513 can be a molded piece, a machined piece, a lithographically formed piece, or a combination of these. Angled indent 526 represents a three-dimensional component of the piece (covering) which is a planned feature of the molded piece. Layer 524 is an optional layer which can be deposited on the angled indent 526. Layer 524 can be reflective, refractive, absorbing, or diffusing, having a different index of refraction from the covering 513 material. Diffuser 528 is another feature which can optionally be built into the molded covering 513. Diffuser 528 is a feature adapted and configured to further direct, focus, diffuse, or otherwise condition the radiation leaving source 502. One or more projections 530 can be deposited or glued onto covering 513. These projections 530 can be adapted and configured to enhance heat transfer, enhance bonding, or enhance conduction to an underlying mount. Although covering 513 depicts space for only one set of radiation sources 522, those skilled in the art will recognize that more than one radiation source or sources can be included in covering 513.

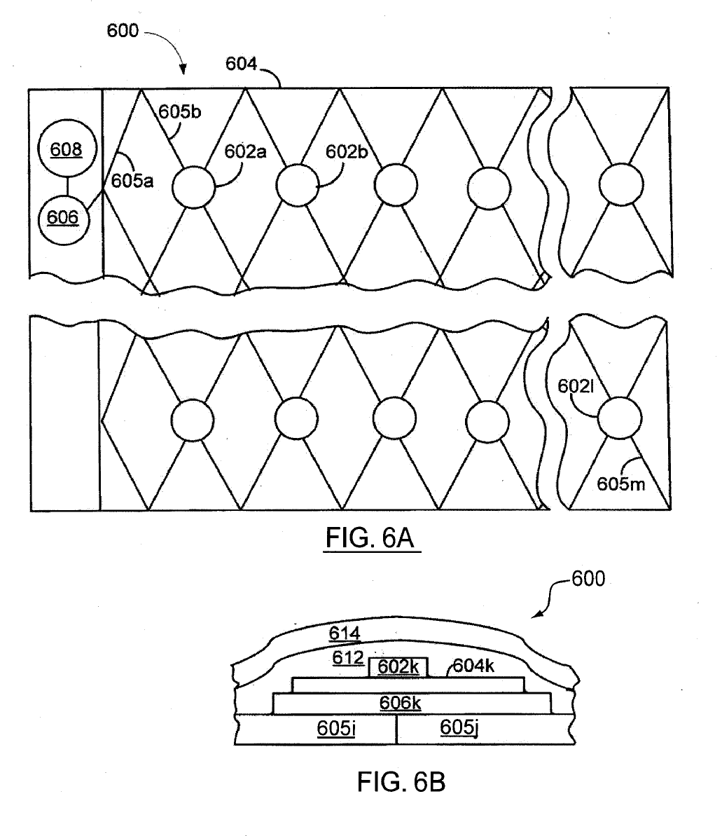

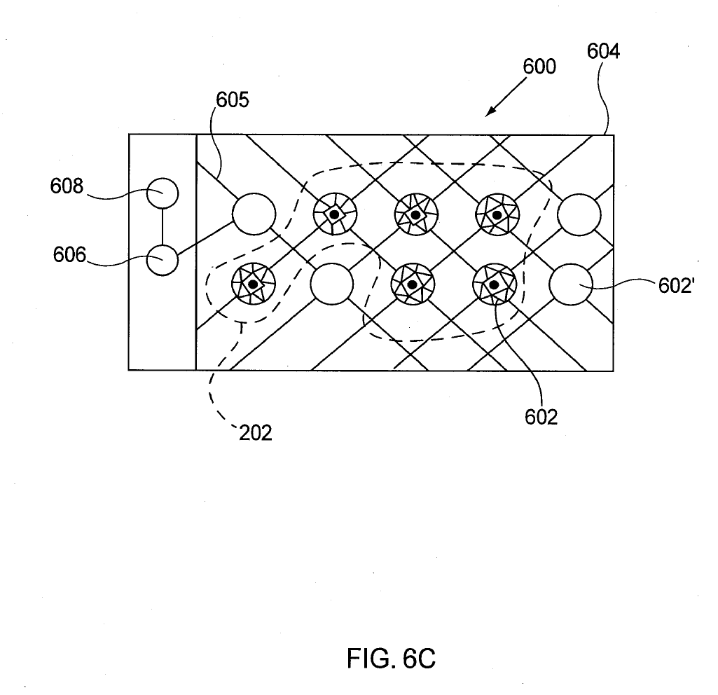

FIG. 6A shows a radiation applicator 600. Radiation applicator 600 includes radiation source(s) 602a-l, substrate 604 having cords 605a-605m, controller 606, and power source 608. In other embodiments, radiation applicator 600 may not have all of the components in FIG. 6A or may have other components in addition to, or instead of, those in FIG. 6A.

Radiation applicator 600 may be an embodiment of radiation applicator 100. Radiation source(s) 602a-l could be of any of the types of radiation source(s) as radiation source(s) 102 (e.g. 102a-102n) and/or 500. Substrate 604 may be a mesh (e.g., a flexible net) that is made of crisscrossing cords 605a-m, which may be an embodiment of substrate 104 in FIG. 1. For example, the flexible net that makes up substrate 604 may be a bandage which is highly elastic. Radiation source(s) 602a-l can be placed at the intersection of individual cords 605a-m of substrate 604. In an alternative embodiment, radiation source(s) 602a-602l may be placed on other parts of cords 605a-605m in addition to, or instead of, being placed at the intersections of two of cords 605a-605m. Controller 606 may be the same as controller 304, and power source 608 may be the same as power source 304. Cords 605a-m may carry or may include electrical connections 306 and/or optical fibers that bring electricity and/or optical communications from controller 606 to radiation source(s) 602 for powering and/or communicating with radiations sources 602a-602l. The configuration of cords 605a-605m allow radiation source(s) 602a-602l to cool by allowing air to pass across the backs of radiation source(s) 602a-602l. The configuration further allows for flexible spacing between the intersections of the cords. In this way, the material (the nodes) can be spread apart by applying force to the edges of the radiation applicator 600 and then allowed to return to the prior spacing when the edges are allowed to return their previous spacing. Although the embodiment of FIG. 6A does not include a region such as region 106, in an alternative embodiment, substrate 604 may include a region 106.

FIG. 6B shows a cross-section of an example of an embodiment of a radiation applicator 600. The embodiment of FIG. 6B includes light source 602k, mount 604k, cord 605i, cord 605j, header 606k, spectral conditioner 612, and optional patient interface 614. In other embodiments, radiation applicator 600 may not have all of the components associated with FIG. 6B and/or may have other components in addition to, or instead of, those associated with FIG. 6B.

Light source 602k, mount 604k, and header 606k are the light source, mount, and header of one of radiations sources 602a-602n. Light source 602k, mount 604k, and header 606k may be embodiments of light source 502, mount 504, and header 506, respectively. Similarly, spectral conditioner 612 and optional patient interface 614, which may include adhesive, may be an embodiment of spectral conditioner 512 and optional patient interface 514, respectively. Cords 605i and 605j are two of cords 605a-605l. Cords 605i and 605j are a pair of cords that criss-cross one another under mount 604k.

As discussed above, the radiation applicator 600 can be adapted to be placed on a patient at a target body surface such that it covers, or substantially covers, a therapeutic surface area. As shown in FIG. 6C, the radiation applicator 600 is applied to the target body surface such that the radiation applicator 600 covers a lesion 202, to which therapy will be delivered. Further radiation sources 602, 602′ associated with the radiation applicator 600 can be selectively activated such that a first subset of radiation sources (602) is on, while the remainder of the radiation sources (602′) are not on. As illustrated, the first subset of radiation sources 602 are positioned within the radiation applicator 600 such that the radiation sources 602 can apply therapy to the lesion 202. As will be appreciated by those skilled in the art, the first set of radiation sources 602 can be further divided into subsets that are separately programmable to deliver different therapeutic doses. This embodiment would be appropriate where, for example, a lesion to be treated has, within the lesion, areas that require more therapeutic treatment than other areas (e.g., a border region of a lesion might require less therapy, than a central portion).

### EXAMPLE 1

UV LED (310 nm) chips from S-ET (Columbia, S.C.) were used as one radiation (light in this case) source 102a; the chip was mounted on a gold patterned aluminum nitride sub-mount and further mounted on a Kovar header. The patterning was performed by Advanced Thin Film, Fremont, Calif. The chips were bonded to a eutectic metal layer (e.g. gold-tin alloy) which was deposited on parts of the gold portions of a sub-mount and then to a TO-46 (well-known to those skilled in the art) header package. An aluminum reflector was attached with epoxy to the header which serves to reflect the light toward the spectral conditioner (e.g. a lens in this case). In this example, a patient interface was not included in the assembly. The radiation applicator was then applied to the skin for approximately 14 minutes. At 14 minutes, the dose was sufficient to sunburn a region of one square centimeter (the region had previously been assessed to require 350 mJ/cm² for an MED at 310 nm). The required voltage was approximately 4.5 Volts and the current approximately 35 milliamps; the required optical power was therefore 340 microwatts per cm² (energy over time) which translates to 150-200 milliwatts of electrical power. A collection of portable alkaline batteries (AA) was used for this experiment. However, many different battery sources can be utilized. Any power supply can be used which generates the appropriate currents. As discussed above, the power supply only need generate 5-100 mA because only certain ones or groups of LEDs are turned on at a given time. Many common types of power sources can be used for this power regime. For example, batteries such as AA, AAA, B, C, D, 9V, Lithium, Lithium Ion, Zinc-Carbon, Alkaline batteries, rechargeable alkaline batteries, Nickel-Cadmium batteries, Nickel-Metal Hydride batteries, Nickel, Iron batteries, Nickel-Zinc Fuel cell batteries, polymer batteries, photovoltaic batteries, or any other batteries available. Thus, a radiation applicator produced from these LED chips could include many of these devices wired on a substrate. A region such as a 0.5% body surface area (approximately 50 cm²) could be radiated with 50 chips (for example) and utilize 7-10 watts of power over 15 minutes. 5-10 Watts of power could be contained in a portable battery pack and when spread over 50 square centimeters, the generated heat would be dissipated without a large increase in temperature.

### EXAMPLE 2

In another example, a radiation applicator was made using the materials and techniques described with respect to Example 1. However, a UV LED submount containing four LEDs was used for radiation source(s) 102 (e.g. 102a-102n) instead of the UV LED chips. This alternate radiation source 102 resulted in a sunburn over an area of 2 cm² in approximately 3.5 minutes with a voltage of approximately 6 Volts and using 80 milliamps of current.

In applying the device of Example 2 to, for example, psoriasis, which has an average size of a diseased area of approximately 100 cm² (approximately 1% of the body surface area of a patient), is capable of giving a patient a sunburn over a 100 cm² area by applying a therapeutic dose via the 2 cm² sized radiation applicators to 50 2 cm² patches, one patch at a time, each for 3.5 minutes. Radiation applicator 100 is capable of delivering a therapeutic dose of ultraviolet light to these 50 patches, in approximately 50×3.5 minutes=175 minutes, or three hours. This therapy is made possible by controller 302, which directs current to the individual packaged LEDs and ensures that each area of skin receives a therapeutic dose but not more than the therapeutic dose of radiation therapy. In this example, the output from the battery source is approximately 0.5 to 1.0 Watts which is easily accommodated by any of the power sources mentioned above or by a photovoltaic source.

### EXAMPLE 3

In this example, a covering (e.g. 513 in FIG. 5D) was applied directly over the UV LED 502. The silicone used, RTV615, available from GE Silicones was chosen for its patient compatability and its index of refraction which is well-matched to the surface of the LED 502. With this configuration, when the experiment in example 1 was repeated, a sunburn would be achieved in 8 minutes, indicating that the optical output was 612 micro Watts for this experiment, which is a 1.8 fold increase over experiment 1. The resulting device was more comfortable to wear. This example illustrates the manner in which covering 513 can be used to enhance optical output of the radiation source/s 502 in addition to improving the interface between the applicator and the body surface. As described above for FIGS. 5D-E, additional structural features can be included on covering 513 and mount 505 which can further enhance the radiation output from the applicator.

In another embodiment, radiation applicator 100 is constructed from 50 or more radiation applicators 100 of the second example or from one radiation applicator 100 that is similar to that of the second example but 50 times larger. Consequently, either of these larger versions could treat an average sized psoriasis patch of skin in less than 175 minutes (e.g., 3.5 minutes). Furthermore, the efficiency of the LEDs can be expected to improve with time as is well-known to those skilled in the art. With improved efficiency, fewer LEDs may be required for the same clinical effort.

FIGS. 7A-B illustrates another embodiment of an optical therapy device for treating a target surface where the target surface is the eye. The device is adapted to treat disorders of an external surface of an eye (e.g., allergic conjunctivitis). Allergic conjunctivitis is a common clinical problem and there are few therapies that are well accepted. Immunosuppressive regimens, which involve the use of tacrolimus, a macroline lactone or calcineurin inhibitor (see, Joseph et al., Topical Tacrolimus Ointment for Treatment of Refractory Anterior Segment Inflammatory Disorders, 24(4) Cornea 24417-20) has been used to treat atopic keratoconjunctivitis, chronic follicular conjunctivitis, and blepharokeratoconjunctivitis. Ultraviolet light may be used to treat allergic conjunctivitis by providing a local therapy to suppress the inflammatory response and immune reaction against the offending antigen. The optical therapy device for the eyes is generally configured to prevent ultraviolet rays from affecting the patient's lens or retina. Other disease states, including dry eyes, have also been shown to respond to immunosuppressive drugs such as cyclosporine (see, Tang-Liu, et al., Ocular Pharmacokinetics and Safety of Cyclosporine, a Novel Topical Treatment for Dry Eye, 44(3) Clin Pharmacokinetics 247-61 (2005)). Other phototherapeutical modalities, such as intense pulsed white light, high intensity blue light can also be used to treat dry eye and allergic conjunctivitis.

In some embodiments, the optical therapy device 700 is used with a slit lamp to treat patients with allergies such that only the sclera 735 (see FIGS. 7A-B, the portion of the eye affected by the conjunctivitis) absorbs the light (specifically ultraviolet light) and the lens and the retina do not. The light (UV or white light) is essentially focused onto an area 736 having a hole 738 or region without light in the center. FIG. 7b depicts the projection of the light conditioned through the slit lamp as it would appear on a flat surface. The hole 738 in the center generally corresponds to the location where light enters the eye; selective targeting of the light to region 736 allows this region to be excluded from the optical therapy. In another embodiment (FIG. 7A), a contact lens is provided to create region 736. In the case of the contact lens, a beam of ultraviolet light can be used which does not have a UV sparing region in its center. In such an embodiment, the contact lens creates the region 736 wherein the pupil region is excluded from the ultraviolet light.

FIG. 8A depicts another embodiment of the therapeutic device 800 of the current invention applied to the body surface of a patient (skin in this case) 842. Device 800 is a probe which delivers phototherapy to a patient body surface. It can be used synergistically with any of the devices above. The optical output is similar to any or all of the devices depicted above and can be narrow-band, broad-band a combination of narrow band and broadband (for different wavelength regions), or a combination of multiple narrow-band, and/or broadband, and/or low or high power white light. The light sources are any of the light sources in any of the configurations described above. In one example, the light sources are solid state light sources which, as described above, are easily portable by the patient and are powered with a battery pack. The dose of the therapy can be programmed into an integrated microcontroller by a physician before the optical therapy device is dispensed. The radiation sources are used singly or in combination. In one embodiment, the radiation sources are positioned at the distal end 816 of the probe 800. The probe can also include a tip 826 which can be purely passive (for example, a transmissive sheath) or the tip 826 can alter the light output in some way (for example, a diffusive tip). The output of the probe 800 in each spectral region can be controlled so that some radiation sources are off while others are on. For example, although a radiation source is placed at the end of the probe 800, if one radiation source on the chipset is activated, the output of the probe will be only the radiation from the radiation source; or if additional radiation sources are included on the probe, then the output will be the output of the multiple radiation sources. Furthermore, the probe can fit into a bandage to illuminate a target region through the bandage.

The therapeutic device 800 can also be used in conjunction with any of a multitude of moieties as a photodynamic therapy device as described above and in U.S. patent application Ser. No. 11/152,946. Device 800 in FIG. 8 can also be used in conjunction with the wearable devices above. The diseases of the skin which can be treated with the therapeutic device 800 include but are not limited to: vitiligo, psoriasis, atopic dermatitis, mycoses fungoides (T-cell lymphomas), skin cancers, and infections (e.g., fungal infections). The device 800 may also contain integrated photodetectors, which can continuously readjust the device's output or can detect a disease state of the skin so that the optical therapy can be applied. Device 800 can also incorporate any of the heat conducting features of devices in U.S. patent application Ser. No. 11/152,946 to which this application claims priority, as well as any of the features described in U.S. patent application Ser. No. 11/304,824 filed Jan. 25, 2006 by Gertner et al. for Optical Therapy Devices, Systems, Kits and Methods for Providing Therapy to a Body Cavity.

FIG. 8B shows another embodiment of the therapeutic device 800 in which radiation sources are incorporated into a device which can be worn or otherwise fixtured, carried, or attached to a patient while the therapy to treat a skin disorder is being applied. Although the device 800 of the embodiment illustrated in FIG. 8B has the form of a bracelet, the radiation sources 840 can be incorporated into any material which can at least partially cover or are in direct or indirect contact with the patient's skin 842. For example, the therapeutic device 800 may have the form of a bandage, blanket, any articles of clothing, a ring, jewelry, a hat, a wristband, a shirt, a sock, underwear, a scarf, a headband, a patch, a gauze pad, or any other wearable article, etc.

In another embodiment, several devices 800, 100 (e.g., bandages) are brought together or applied to treat a larger area. In one embodiment, a kit having different sized bandages is provided. Adhesive can be a component of the kit and/or a component of the bandages. The individual sized bandages can be fit together to irradiate different shaped and sized areas or lesions. With such a “wearable” device 100, a patient can treat his or her disorder (e.g., psoriasis) while performing other tasks or sleeping and can treat small or large areas of disease in a time- and cost-effective manner.

Such a localized therapy is also safer than treatments which apply light over a broad area of skin because portions of the skin which are not psoriatic can be unnecessarily exposed to ultraviolet light. With the LED systems described above, broad-band or narrow-band optical therapy can easily be applied to the skin depending upon clinical requirements. In addition, photodetectors may be integrated into the therapeutic device 100 for feedback control of the therapy. Internal body cavities can be treated as well with permanent or semi-permanent optical therapy devices 100. For example, in one embodiment, inner ear infections are treated by placing an optical therapy device 100 inside or proximal to the ear canal.

FIG. 8C illustrates an optical therapy device 800 being applied to a finger or toe nail. In such a case, tinea infections of the nails may be treated with the device by choosing appropriate optical wavelengths (e.g., 255-320 nm) for the radiation sources. FIG. 8D illustrates an optical therapy device 800 used to treat fungal infection of the nail beds 444. The optical therapy device 800 has the form of a bandage or Band-aid®. Such a device 800 allows patients to go about their daily lives while the treatment is being applied. The device 800 is constructed using the principles and methods describes above. Device 800 can be used in combination with photosensitizers or photodynamic agents to better treat the nailbed. In another embodiment, the device shown in FIGS. 8C-D are used to treat nail psoriasis in which case wavelengths between 295 nm and 320 nm, typically would be used.

The devices and radiation source(s) disclosed herein can be used for therapies such as psoriasis or other skin disorders currently treated with radiation (e.g., vitiligo, cutaneous T cell lymphoma, fungal infections, etc.). The preferred action spectrum to treat psoriasis is approximately 308-311 nm. In addition, narrow-band radiation is generally more effective than broad-band radiation. One limiting factor in current modalities and technologies for the treatment of psoriatic lesions is that typical devices available on the market today are large and expensive, and generally require patients to visit a physician's office for treatment. Home-treatment devices are typically large fluorescent lamps that are adapted to treat a broad area rather than a localized region. Whether in the home or in the office of the medical practitioner, the therapy takes time out of the patient's daily schedule. In addition, it is typically difficult for a patient to perform other tasks while the therapy is being applied. Furthermore, with current technology, it is difficult to treat a small area of the skin with narrowband light. Lasers are sometimes used to do so, but lasers are generally expensive and are not practical as home-based therapy devices.

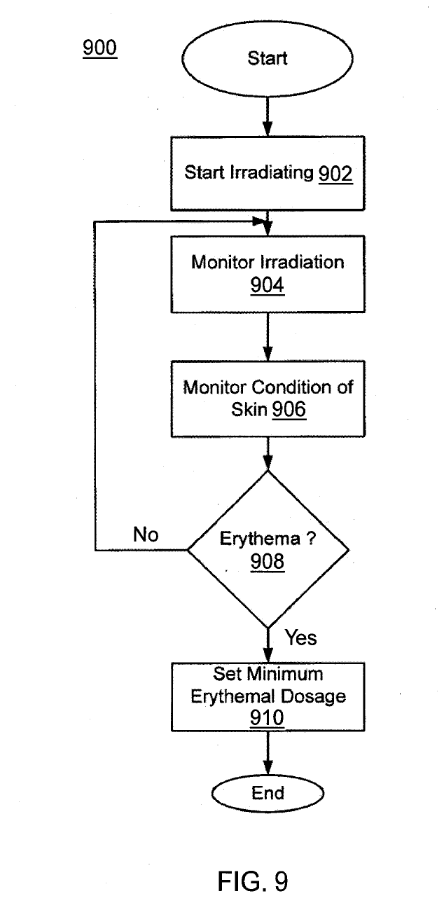

FIG. 9 shows a flowchart of an example of a method 900 for calibrating radiation applicator 100 for a particular patient and disease. Method 900 may be an embodiment of program 408. In step 902, system 900 begins to irradiate a target portion of a body that is not affected by the disease being treated. Step 902 may include placing batteries in power source 304 and turning on system 304. Step 902 may also include entering a command into controller 302 that places radiation applicator 100 in a calibration mode and/or turns on radiation applicator 100. In step 904, the irradiation is monitored by keeping track of how long the target portion being irradiated has been irradiated and/or how much radiation is being sent to the target portion. Step 904 may be implemented by timing how long each of the radiation source(s) 102 (e.g. 102a-102n) have been kept on and/or by actually measuring samples of the output from radiation applicator 100. In step 906, the condition of the portion being irradiated is monitored. The monitoring may be performed by a detector or by a human being periodically checking the condition of the target portion being irradiated. Steps 902, 904, and 906 may be initiated in any order. The actual monitoring of the condition of the target portion is performed while irradiating the target portion or by interrupting the irradiation.

In step 908, based on the monitoring of step 906, a decision is made as to whether the portion being irradiated has received an erythemal dose (or other indicator of toxicity and/or efficacy in any other disease to be treated). In an embodiment, during step 908, the first detectable point when the portion being irradiated has received an erythemal dose triggers a decision that an erythemal dose was reached. If an erythemal dose has not been received, steps 902, 904, and 906 are allowed to continue to be performed. If an erythemal dose has been received, method 900 proceeds to step 910. Steps 902, 904, 906, and 908 may be implemented by applying escalating doses of radiation over a specific period of time (e.g., 24 hours) to different regions of a body surface. After each period of time, if erythema is not reached, the period of time is started over again, but the dose of radiation delivered is escalated (e.g., increased) to a higher amount. If, after a given period, erythema is reached, the method proceeds to step 910. In step 910, the MED is determined, and radiation applicator 100 is configured so that when used in the future, radiation applicator 100 will deliver a MED or a somewhat lower dosage; in any case, the MED acts as an internal calibration for each patient's skin type. In some therapeutic regimens, multiples of the MED is required or fractions of the MED may be required for therapy. The MED is determined by comparing the results of steps 904 and 906. The MED may be expressed as a period of time for running radiation applicator 100 and optionally by recording information about the duty cycle used. Alternatively, the total amount of time that each individual one of radiation source(s) 102 (e.g. 102a-102n) was left on is recorded. Step 910 may be just recording the MED on paper or may be entering the MED into memory 404, entering a setting into controller 302 that determines either how long radiation applicator 100 stays powered on before controller 302 shuts off radiation applicator 100 or the total time that any one of radiation source(s) 102 (e.g. 102a-102n) is allowed to be on.

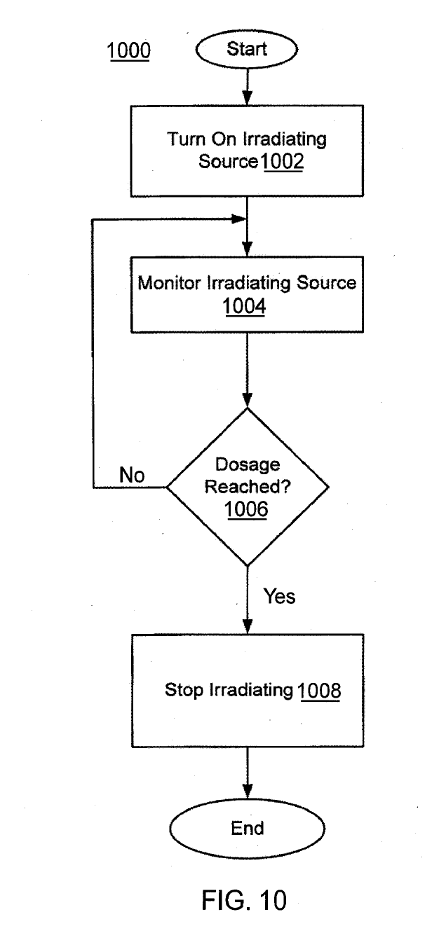

FIG. 10 shows a flowchart of an example of a method 1000 of using radiation applicator 100. Method 1000 may be an embodiment of program 406. In step 1002, radiation applicator 100 is turned on, and radiation applicator 100 irradiates a portion of a target portion of a body. Step 1002 may include sending current via electrical connections 306 from power source 304 to controller 302. Step 1002 may include processor 402 running program 406, which causes signal generator 405 to generate signals. Step 1002 may also include controller 302 sending the generated signals via electrical connections 306, thereby turning on some of radiation source(s) 102 while turning off others of radiation source(s) 102 according to a cycle that is based on a maximum amount of power that power source 304 can support. In step 1004, the irradiation is monitored. Step 1004 may include timing how long radiation applicator 100 has been on and/or turning how long each radiation source(s) 102 and/or each group of radiation source(s) 102 have been on. Alternatively, step 1004 may involve sampling and measuring some of the output of radiation applicator 100 and/or the condition of the target portion in addition to, or instead of, timing how long radiation applicator 100 has been on. In step 1006, a determination is made as to whether a prescribed dose has been received and/or delivered. If a prescribed dose has not been received or delivered, steps 1002 and 1004 are allowed to continue. If a prescribed dose has been received or delivered, then method 1000 continues to step 1008, where radiation applicator 100 the irradiation of the target portion is terminated. Since different radiation source(s) 102 (e.g. 102a-102n) are on at different times, different ones of radiation source(s) 102 (e.g. 102a-102n) deliver the prescribed dose at different times. Consequently, step 1008 may involve selectively turning off individual ones of, or groups of, radiation source(s) 102 that have delivered the prescribed dose or that are over areas where the prescribed dose has been received. After step 1008, method 1000 terminates. Method 1000 may be implemented by turning on radiation applicator 100, by setting a timer that shuts down each of radiation source(s) 102 of radiation applicator 100 after a period of time has elapsed during which it is expected that the radiation source has delivered a dose as determined by the patient or medical practitioner.

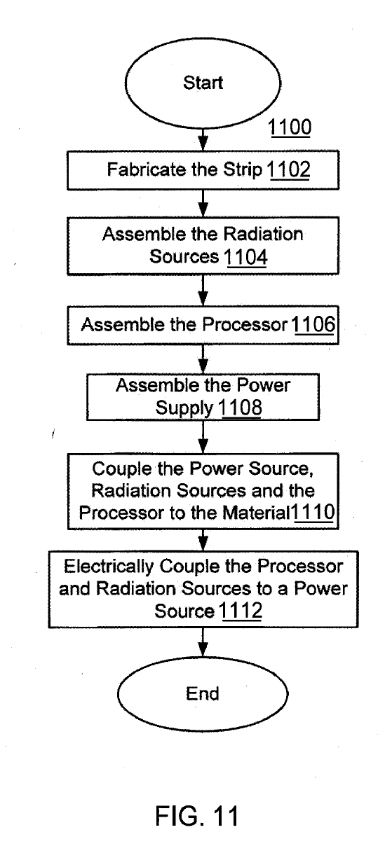

FIG. 11 is a flowchart of a method 1100 of assembling radiation applicator 100. During step 1102, radiation source(s) 102 are assembled. During step 1104, substrate 104 is fabricated. During step 1106, controller 302 is assembled. During step 1108, power source 304 is assembled. During step 1110, radiation source(s) 102, controller 302, and power source 304 are coupled to substrate 104. Step 1110 may include attaching radiation source(s) 102, controller 302, and power source 304 to region 106. During step 1112, controller 302 and radiation source(s) 102 are communicatively coupled to one another. For example, connections 306 are placed on substrate 104 and connected to controller 302, power source 304, and radiation source(s) 102. Steps 1102, 1104, 1106, and 1108 may be performed simultaneously or in any order with respect to one another. Similarly, steps 1110 and 1112 may be performed simultaneously or in any order with respect to one another.

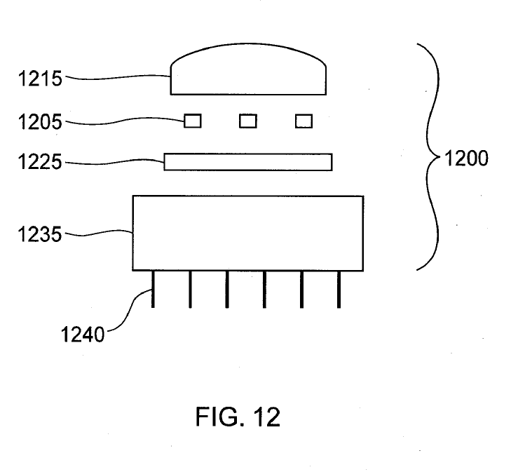

In one embodiment, the radiation devices are light emitting diodes (LEDs) and the material between the LEDs and the covering which interfaces with the body surface is transparent to the light emitted from the LEDs. In one embodiment, the LEDs emit ultraviolet light in the wavelength range 250-365 nm. The LEDs are chips which are then assembled into modules, or radiation sources” which can be manipulated into a larger device. FIG. 12 depicts a radiation source 1200 which consists of LED chips 1205, a chip covering 1215, a chip submount 1225 and a base 1235 (often referred to as a header in the industry).

The base 1235 can be produced from a substance with a high coefficient of heat transfer to conduct heat away from the chips and the skin. The base can be microfabricated, molded, or machined. The base can further be shaped to conduct heat in an optimal manner. For example, fins 1240 can be fabricated, deposited, or glued onto the base. In another embodiment, a thermoelectric cooler is attached to the base. The base can further be processed such that it fits into a circuit on the irradiating device 100. In this embodiment, the substrate of the radiative device 100 is made so that the base (and module) can easily press-fit into the radiating device. The portable irradiating device then has contacts thereon which provide for electrical communication between the controller and the module 1200.

Covering 1215 is made from a material transparent to the radiation emitted from the device. In the case where the chips 1205 emit ultraviolet radiation, the covering 1215 can be produced from a material such as silicone or fluorinated-ethylene propylene (FEP). It is preferable that the covering 1215 optically match the surface of the chip so (as described in Example 3, above), so as to minimize reflection and loss of photons at the interface of the two materials. Covering 1215 can further contain additional interfaces which serve to condition the light as it leaves the chips. For example, a covering material (e.g. epoxy, silicone, quartz, FEP) can have interfaces to diffuse light. In a preferred embodiment, the LED is an ultraviolet LED which emits light from a surface with dimension of about 1 mm squared or smaller. The covering conditions the light so that the light is diffuses over an area of at least 1 cm² from the mount 1225. In another embodiment, the covering conditions the light so that the light diffuses over an area of between 0.4 cm² and 1 cm² from the smaller mount. In another embodiment, one chip diffuses light to an area less than 4 cm². In yet another embodiment, the covering conditions the light to spread over an area greater than 1 cm². The conditioned light need not be uniform or even close to uniform. When 1-2 cm² (for example) is used, the covering 1215 can diffuse light from a mount less than about 1-3 mm² to a region 1-2 cm² over a distance of between 0.5 and about 5 mm (the distance between the LED devices and the skin).

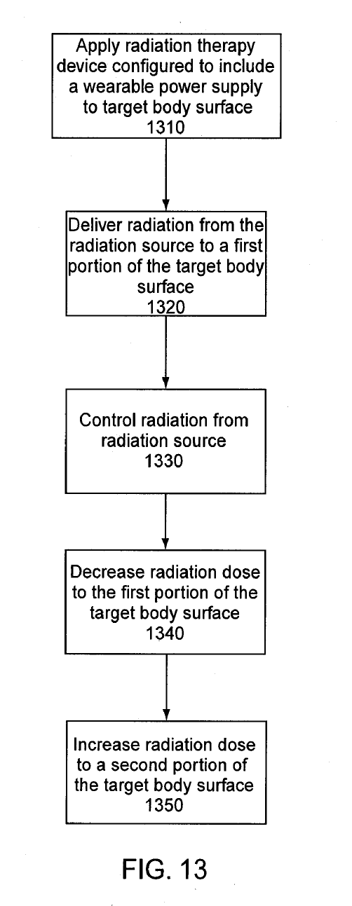

FIG. 13 illustrates the steps of a method according to one embodiment of the invention for treating a target body surface. Initially a radiation therapy device adapted and configured to include a wearable power supply, a radiation source, and a controller to the target body surface is applied to the body 1310. Thereafter, radiation from the radiation source is delivered 1320 to a first portion of the target body surface. The radiation output is controlled 1330 from the radiation source to a first portion of the target body surface in relation to a therapeutic dose during the step of delivering radiation. By controlling the output, the radiation dose can be decreased 1340 to the first portion of the target body surface. Additionally, by controlling the output, the radiation dose can be increased 1350 to a second portion of the target body surface. The method can be performed using a suitable radiation source such as an LED, or a UV LED. In some embodiments the method is performed by relating the therapeutic dose to minimal erythema dose. Additionally, the step of decreasing the radiation dose can include turning-off the radiation dose; similarly, the step of increasing the radiation dose can include turning on the radiation dose. The radiation therapy device can be a wearable article. Further, the step of delivering radiation can include delivering a timed radiation dose. The radiation dose can be delivered to any portion of the target body surface between 100 mJ/cm and 3 J/cm². In some instances, it may be desirable to deliver radiation doses to a portion of the target body surface at a wavelength between 295 and 315 nm. In other embodiments, the radiation dose delivered to any portion of the target body surface has a first wavelength between 295 and 315 nm and a second wavelength between 340 and 400 nm. In still other embodiments, the radiation dose delivered to any portion of the target body surface has as wavelength greater than about 700 nm. The method can also include the step of administering a photosensitizing agent. Additionally, the method can include the step of programming the radiation device to apply radiation to a prescribed area. In some embodiments, the method can further comprise the step of programming the radiation device to turn off after a prescribed dose is applied. Additionally, the step of monitoring the radiation device can be included in the method, as desired. The radiation dose delivered to any portion of the target body surface can be selected from: infrared light, intense pulsed light, white light, and combinations thereof.

A variety of kits are also contemplated for use with this invention. For example, patients could be provided with kits that have a plurality of radiation applicators with different sizes and shapes and in which each size and shape can be fit together. The applicators could be configured to provide the same radiation for the same amount of time, or could be applicators having different radiation types and/or amounts and/or time configurations. The applicators can be fit together and then further adapted to communicate with a computer program to customize the type, quality, quantity and/or location of treatment to a pre-defined region. For example, where it would be desirable to provide a first quality of treatment at a first time and a second quality of treatment at a second time, or where it is anticipated that the amount of radiation and/or time of radiation required would change during the course of delivering the therapy. Thus, for example, a first radiation applicator having the ability to deliver a first amount of radiation at a first amount of time, could be provided with a second radiation applicator having the ability to deliver a second amount of radiation for a second amount of time. Thus enabling a kit to be provided that has the ability to slowly increase therapy over time, increase and then decrease therapy over time, or decrease therapy over time.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A phototherapeutic treatment apparatus comprising:
(a) a first light source adapted to emit light at a peak wavelength between about 250 nm and about 320 nm;
(b) a second light source adapted to emit light at a peak wavelength; a support structure adapted to be applied to a patient and further adapted to support the light sources in a manner permitting light from the light sources to reach a prescribed body surface of the patient;
(c) a controller adapted to automatically or semi-automatically operate the light sources within safe and therapeutic limits; and
(d) a power supply adapted to provide power to the light sources through the controller.

2. The phototherapeutic treatment apparatus of claim 1 wherein the first or second light source is adapted to deliver light in relation to a minimal erythema dose.

3. The phototherapeutic apparatus of claim 1 wherein the first or second light source is programmable through the controller and the controller adapted and configured to control light delivery by the first or second light source to a prescribed area.

4. The phototherapeutic treatment apparatus of claim 3 wherein the controller is adapted and configured to turn off the first or second light source after delivery of a prescribed dose.

5. The phototherapeutic treatment apparatus of claim 3 wherein the controller is adapted and configured to monitor a radiation dose delivered by the phototherapeutic treatment apparatus.

6. The phototherapeutic treatment apparatus of claim 1 wherein the light source is adapted and configured to deliver light, the light being selected from the group consisting of UVA, blue, yellow, white, and infrared light.

7. A phototherapeutic treatment apparatus comprising:
(a) a light source adapted to emit light at a peak wavelength between about 250 nm and about 320 nm;
(b) a support structure adapted to be externally applied to a patient and to support the light source in a manner permitting light from the light source to reach a target body surface of the patient; and
(c) a controller adapted to operate the light source; and a wearable power supply adapted to provide power to the light sources.

8. The phototherapeutic treatment apparatus of claim 7 wherein the light source is adapted to deliver a dose related to the minimal erythema dose.

9. The phototherapeutic treatment apparatus of claim 7 wherein the light source is a programmable light source adapted and configured to deliver light to a prescribed area.

10. The phototherapeutic treatment apparatus of claim 9 wherein the programmable light source is adapted and configured to turn off after delivery of a prescribed dose.

11. The phototherapeutic treatment apparatus of claim 9 wherein the programmable light source is adapted and configured to monitor the radiation device.

12. The phototherapeutic treatment apparatus of claim 7 further comprising a second light source, the second light source adapted and configured to deliver light, the light being selected from group consisting of UVA, blue, yellow, white, and infrared light.

13. The phototherapeutic treatment apparatus of claim 7 wherein the light source is adapted and configured to deliver intense pulsed white light.

14. The phototherapeutic treatment apparatus of claim 7 wherein the light source is adapted and configured to deliver intense pulsed white light in combination with infrared light.

15. A method of treating a prescribed area of a target body surface comprising the steps of:
(a) applying a radiation therapy device adapted and configured to include a wearable power supply, a radiation source, and a controller to the target body surface;
(b) delivering a therapeutic dose of radiation to at least a portion of the target body surface; and
(c) controlling a radiation output from the radiation source to a first portion of the target body surface in relation to a therapeutic dose during the step of delivering radiation.

16. The method of claim 15 wherein the radiation source comprises an LED.

17. The method of claim 16 wherein the radiation source comprises an LED selected from the group consisting of LEDs adapted to deliver UVA, blue, yellow, white and infrared light.

18. The method of claim 16 wherein the LED comprises a UV LED with a peak wavelength between 250 nm and 320 nm.

19. The method of claim 18 wherein the wherein the radiation source is adapted and configured to deliver light, the light being selected from group consisting of UVA, blue, yellow, white, and infrared light.

20. The method of claim 18 wherein the therapeutic dose delivered to any prescribed area of the target body surface is between 1 mJ/cm² and 3 J/cm².

21. The method of claim 15 wherein the therapeutic dose is related to a minimal erythema dose.

22. The method of claim 15 wherein the step of controlling the radiation output includes powering off the radiation source.

23. The method of claim 15 wherein the step of controlling the radiation output includes powering on the radiation source.

24. The method of claim 15 wherein the radiation therapy device is a wearable article.

25. The method of claim 15 further comprising the step of programming the radiation device to apply radiation to a prescribed area.

26. The method of claim 15 wherein the radiation output delivered to any portion of the prescribed area of the target body surface has a wavelength between 295 and 315 nm.

27. The method of claim 15 wherein the radiation dose delivered to the prescribed area of the target body surface has a peak wavelength between 340 and 400 nm.

28. The method of claim 27 wherein the radiation source comprises an LED selected from the group consisting of LEDs adapted to deliver UVA, blue, yellow, white and infrared light.

29. The method of claim 15 wherein the radiation dose delivered to the prescribed area of the target body surface has as wavelength greater than about 700 nm.

30. The method of claim 29 wherein the radiation source comprises an LED selected from the group consisting of LEDs adapted to deliver UVA, blue, yellow, white and infrared light

31. The method of claim 15 further comprising the step of administering a photosensitizing agent.

32. The method of claim 15 further comprising the step of programming the radiation device to turn off after a prescribed dose is applied.

33. The method of claim 15 wherein the radiation dose delivered to the prescribed area of the target body surface is intense pulsed white light in combination with infrared light.

34. A phototherapeutic treatment device comprising: a plurality of light sources each adapted to emit light at a peak wavelength; a support structure adapted to be applied to a patient and further adapted to support the light sources in a manner permitting light from the light sources to reach a prescribed body surface of the patient; and a controller adapted to variably activate a subset of the plurality of light sources.

35. The phototherapeutic treatment device of claim 34 wherein the subset of activated light sources is adjacent a body surface to be treated.

36. The phototherapeutic treatment device of claim 34 wherein the subset of activated light sources at a first point in time is different than an activated subset of light sources at a second point in time.

37. The phototherapeutic treatment device of claim 34 wherein the light sources are configured to emit light at more than one peak wavelength.

38. The phototherapeutic treatment device of claim 34 wherein the plurality of light sources comprise a first group of light sources adapted and configured to deliver light at a first peak wavelength and a second group of light sources adapted and configured to deliver light at a second peak wavelength.

39. A kit for delivering therapy to an eye comprising: a contact lens with a light blocking portion; a phototherapeutic treatment apparatus comprising a light source adapted to emit light at a peak wavelength; a light source support structure adapted to support the light sources in a manner permitting light from the light sources to reach an area of the patient; and a power supply adapted to provide power to the light sources.

40. The kit of claim 39 wherein said light source emits ultraviolet light and said light blocking portion blocks ultraviolet light.

41. A phototherapeutic treatment apparatus comprising: a first light source adapted to emit light at a peak wavelength; a light source support structure adapted to contact a patient and to support the light sources in a manner permitting light from the light source to reach a target body surface area of the patient; a controller adapted to allow for autonomous operation of the light source within a therapeutic limit.

42. The treatment apparatus of claim 41 further comprising a wearable power supply.

43. The treatment apparatus of claim 42 further comprising a plurality of light sources.

44. The treatment apparatus of claim 43 wherein the light sources are LEDs.

45. The treatment apparatus of claim 44 wherein the controller is adapted to apply a treatment dose to a prescribed region through a software program.

46. The treatment apparatus of claim 41 wherein the controller increases the dose to at least one light source while decreasing the dose to at least one other light source while applying the treatment dose to the specific region.

47. The treatment apparatus of claim 44 wherein at least one LED emit light with a peak wavelength in the range from 250 nm to 320 nm.

48. The treatment apparatus of claim 44 wherein at least one LED emits light with a peak wavelength in the range from 320 nm to 400 nm.

49. The treatment apparatus of claim 44 wherein at least one LED emits light with a peak wavelength in the range from 400 nm to 500 nm.

50. The treatment apparatus of claim 44 wherein at least one LED emits light with a peak wavelength in the range from 600 nm to 700 nm.

51. The treatment apparatus of claim 44 wherein at least one LED emits light with a peak wavelength greater than 700 nm.
